SEQUENCE LISTING (1 sequence)

SEQ ID NO: 1 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKIFEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFFDYRYSQADALKYVGIEREMEIP

(19) United States
(12) Patent Application Publication
(10) Pub. No.: US 2022/0016111 A1
(43) Pub. Date: Jan. 20, 2022

(54) Methods of Treating Inflammation

(71) Applicant: The Walter and Eliza Hall Institute of Medical Research, Melbourne, Victoria (AU)

(72) Inventors: Rhys Allan, Parkville (AU); Christine Keenan, Parkville (AU); Stephan Nutt, Parkville (AU)

(21) Appl. No.: 17/312,371

(22) PCT Filed: Dec. 17, 2019
(86) PCT No.: PCT/AU2019/051386

§ 371 Date: Jun. 9, 2021

(30) Foreign Application Priority Data
Dec. 21, 2018 (AU) 2018904884

(51) Int. Cl. A61K 31/496; C07K 16/40; A61K 31/7105; A61K 45/06; A61K 39/395; A61P 37/08

(52) U.S. Cl. CPC A61K 31/496; C07K 16/40; A61P 37/08; A61K 45/06; A61K 39/3955; A61K 31/7105

(57) ABSTRACT

Methods for treating inflammation, including allergic inflammation, lymphocyte-driven inflammation, and inflammatory conditions such as allergic asthma and allergic lung inflammation, using inhibitors of the polycomb repressive complex 2 (PRC2) methyltransferase enhancer of zeste homolog 2 (Ezh2) are provided.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The entire content of the electronic submission of the sequence listing is incorporated by reference in its entirety for all purposes.

### FIELD

The present disclosure relates generally to the field of inflammation, particularly allergic inflammation and more particularly to the use of Ezh2 inhibitors to treat allergic inflammation, lymphocyte-driven inflammation and non-allergic asthma.

### BACKGROUND

The immune system functions to maintain homeostasis and protect against pathogen challenge. However, sometimes hypersensitivity to usually innocuous antigens results in allergic conditions such as asthma and allergic rhinitis or auto-inflammatory diseases such as lupus and type 1 diabetes. Unfortunately, these disorders are increasing in prevalence and current treatments are mostly aimed at ameliorating the symptoms but not switching off the lymphocytes that cause the disease pathology. Therefore, novel strategies are desperately needed to rewire the immune response in such situations with the aim of perturbing the inflammatory cascade and potentially curing disease.

The major orchestrators of immune cell function in the development of allergic responses are the CD4⁺ T-helper (Th) cells. In animal models of allergic asthma, the presence of CD4⁺ T cells is absolutely required for the induction of airway inflammation and recruitment of eosinophils (Gonzalo et al., (1996) J Clin Invest 98, 2332-2345). Allergen-specific Th cells are activated by dendritic cells in the draining lymph nodes and infiltrate into the lung tissue and secrete cytokine to orchestrate and exacerbate the inflammatory response (Lambrecht, B. N., and Hammad, H. (2011). Annu Rev Immunol). Although a number of Th subsets are known, the main players in promoting this allergic response appear to be the Th2 subset which produce important cytokines such as IL4, IL-5, and IL-13 and chemokine signals that result in the recruitment, activation of the innate immune cells.

The factors that control the relative stability and plasticity of Th phenotypes are critical to immune responses (Allan, R. S., and Nutt, S. L. (2014). Immunol Rev 261, 50-61) and although the signals and transcription factors guiding Th differentiation are well defined, it appears that the heritability through cell division is controlled predominantly by epigenetic mechanisms. Correlations have been observed between levels of several histone-H3 and H4 modifications with activity or silencing of cytokine genes in committed Th1 and Th2 cells (Avni, O., et al. (2002). Nat Immunol 3, 643-651). Importantly, it has been found that stable Th1 gene silencing in Th2 cells requires the expression of the Suv39h1 enzyme to maintain the balance of methylation and acetylation at lysine 9 of histone-H3 (H3K9) in Th1 genes (Allan, R. S., et al. (2012)). Nature 487, 249-253). Furthermore, in a murine model of Th2 allergic asthma, the loss of Suv39h1 resulted in skewing towards a Th1 response and decreased lung pathology (Allan et al., (2012) supra). Targeting enzymes involved in epigenetic modifications to chromatin therefore has the potential to perturb the function of the lymphocytes that drive the allergic response. However, it is unknown whether other epigenetic modifiers or pathways are also involved in the allergic response.

There is a need in the art for strategies that intervene with the development of immune-mediated disorders as current treatments mostly focus on alleviating symptoms rather than reversing the disorders.

### SUMMARY OF THE DISCLOSURE

The present disclosure is based on investigation conducted by the present inventors for epigenetic pathways that are critical for T cells to orchestrate the development of inflammation, in particular allergic inflammation and lymphocyte-driven inflammation. Targeting enzymes involved in epigenetic modifications to chromatin represents an alternative strategy that has the potential to perturb the function of the lymphocytes that drive the immune response. The inventors identified components involved in epigenetic gene silencing that were up regulated after T cell activation and performed in vivo inactivation of these molecules specifically in the T cell lineage. In particular, the inventors found that small molecule inhibition of the PRC2 methyltransferase, enhancer of zeste homolog 2 (Ezh2) reduces inflammation thus representing a novel target for the suppression of inflammatory disorders, and in particular allergic and lymphocyte-driven disorders.

In one aspect, the present disclosure provides a method for preventing, reducing one or more symptoms of, or treating inflammation in a subject, comprising administering to the subject an inhibitor of the polycomb repressive complex 2 (PRC2) methyltransferase enhancer of zeste homolog 2 (Ezh2). In one example, the method prevents, reduces or treats allergic inflammation.

Allergic inflammation may refer to any disorder which is initiated by an allergen. Non-limiting examples of allergic inflammation include allergic asthma, atopic dermatitis, allergic rhinitis (hay fever), urticaria (hives) and food allergies, drug allergies, anaphylaxis and ocular allergic disorders. In one example the method may be used for the prevention, reduction or treatment of early or late phase allergic asthma. In one example the ocular allergic disorder is allergic conjunctivitis.

In another example, the method prevents, reduces or treats lymphocyte-driven inflammation. Non-limiting examples of lymphocyte-driven inflammation include chronic obstructive pulmonary disease (COPD), autoimmune disease, and type-1 diabetes. Preferred examples of autoimmune disease are COPD, systemic lupus erythematosus, type-1 diabetes. Examples of autoimmune disease include lupus (e.g. systemic lupus erythematosus), coeliac disease, acute disseminated encephalomyelitis, acute motor axonal neuropathy, Addison's disease, adiposis dolorosa (Dercum's disease), adult onset still's disease, alopecia areata, ankylosing spondylitis, antiphospholipid syndrome, antisynthetase syndrome, autoimmune pancreatitis, autoimmune thrombocytopenic purpura, autoimmune urticaria, Balo concentric sclerosis, Bechet's disease, Bickerstaff encephalitis, bullous pemphigoid, coeliac disease, chronic fatigue syndrome, chronic inflammatory demyelinating polyneuropathy, cicatricial pemphigoid, Crohn's disease, dermatomyositis, diabetes mellitus type 1, endometriosis fibromyalgia, gastritis, giant cell arteritis, Graves' disease, Graves ophtalmopathy, Guillain barre syndrome, Hashimoto encephalopathy, Hashimoto thyroiditis, juvenile arthritis, lichen planus, Lyme Disease, multiple sclerosis, myasthenia gravis, myocarditis, neuromyelitis optica, pediatric acute-onset, neuropsychiatric syndrome, postmyocardial infarction syndrome, psoriasis, restless leg syndrome, rheumatic fever, systemic lupus erythematosus, scleroderma, Sjogren's, transverse myelitis, ulcerative colitis, vasculitis and vitiligo.

In another example the method prevents, reduces or treats non-allergic (e.g. intrinsic) asthma.

In certain examples, the method of the present disclosure reduces one or more symptoms of allergic inflammation e.g. allergic inflammation. Such symptoms may include one or more of the following selected from itchiness, running nose, sneezing, watery eyes, bronchoconstriction, hives, airway inflammation, anaphylaxis, and dermatitis.

In certain examples, the method of the present disclosure reduces one or more symptoms of lymphocyte-driven inflammation. Such symptoms include one or more of airway obstruction, emphysema, chronic bronchitis. Additionally, lymphocyte driven inflammation can be associated with an increase in pro-inflammatory cytokines (e.g. IL-1β, IL-8, IL-17, TNF, GM-CSF and IFY-γ) and/or Th cells (e.g. Th1 and Th17 cells) which can be measured.

Inhibitors of Ezh2 may be selected from the group consisting of immunoglobulins (e.g. antibodies or antigen-binding fragments thereof), oligonucleotides, ribozymes, aptamers, siRNAs, anti-sense molecules, peptides or drugs (e.g. small molecule inhibitors). In one example, the Ezh2 inhibitor is provided in a pharmaceutically acceptable carrier.

Small molecule inhibitors of Ezh2 are known in the art and are commercially available. Examples of Ezh2 inhibitors that may be suitable for use in the present method include GSK126, GSK343 and GSK503, EPZ005687, EPZ-6438, EI1, CPI-1205. Non-commercial Ezh2 inhibitors that have been described in the literature may also be suitable for the present methods including DzNep, UNC199 and SAH-EZH2 including combinations of any of the foregoing. However, it is to be understood that the Ezh2 inhibitors are not limited to those compounds described above.

In one example, the inhibitor is selective for Ezh2. In other examples, the inhibitor is a dual inhibitor of both Ezh1 and Ezh2. An example of such an inhibitor is UNC1999 (Konze K D et al. ACS Chem Biol. (2013); 8(6):1324-34).

In one example, the Ezh2 inhibitor is administered to the subject in a therapeutically effective amount. The dose administered to the subject may be determined by a physician. In some examples, the dose of Ezh2 may range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

The Ezh2 inhibitor may be administered daily, weekly, bi-weekly, monthly or on an as-needed basis to the subject. In some examples, the Ezh2 inhibitor is administered to the subject in anticipation of an asthmatic or allergic event. In some examples, the Ezh2 inhibitor is administered substantially prior to an asthmatic or an allergic event. As used herein, “substantially prior” means at least six months, at least five months, at least four months, at least three months, at least two months, at least one month, at least three weeks, at least two weeks, at least one week, at least 5 days, or at least 2 days prior to the asthmatic or allergic event.

In some examples, the Ezh2 inhibitor is administered immediately prior to the asthmatic or allergic event (e.g., within 48 hours, within 24 hours, within 12 hours, within 6 hours, within 4 hours, within 3 hours, within 2 hours, within 1 hour, within 30 minutes or within 10 minutes of an asthmatic or allergic event), substantially simultaneously with the asthmatic or allergic event (e.g., during the time the subject is in contact with the allergen or is experiencing the asthma or allergy symptoms) or following the asthmatic or allergic event.

The Ezh2 inhibitor may be administered by any suitable route. In some examples, it is administered by inhalation, ingestion, by a local route (e.g. nasal drops) or by systemic route. Systemic routes include oral and parenteral. Inhaled medications are preferred in some examples because of the direct delivery to the lung, the site of inflammation, primarily in asthmatic patients. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. As used herein, delivery to the nasal passages or the lungs via nasal drops or inhalation are referred to as local administration. Although it is possible that delivery to the lung (e.g., via inhalation) can eventually result in systemic delivery of the agent, the administration is still considered “local” in the sense that the majority of the agent is initially presented to the lung tissue or the nasal passages, prior to any secondary systemic effects. In preferred examples, the Ezh2 inhibitor is administered orally.

For use in therapy, an effective amount of the Ezh2 inhibitor can be administered to a subject by any mode that delivers the inhibitor to the desired surface, e.g., mucosal, systemic.

The Ezh2 inhibitor may be administered as a single agent or in combination with an inhibitor of a protein in the PRC2 protein complex. Examples include inhibitors which target one or more of Suz12, EED, RbAP48, and AEBP2.

In some examples, the Ezh2 inhibitor may be administered in combination with an asthma and/or allergy medicament. Non-limiting examples include antihistamines, theophylline, salbutamol, beclomethasone dipropionate, sodium cromoglycate, steroids, anti-inflammatory agents, IgE antagonists, and anti-IgE antibodies. Such compounds may be administered concurrently or sequentially with the Ezh2 inhibitor.

Preferably, the subject is human. In some examples, the subject is one at risk of developing an inflammation disorder as described herein. In some examples, the subject is one having an inflammation disorder as described herein. In some examples, the subject has asthma. In some examples, the subject has an autoimmune disease.

A “subject having asthma” is a subject that has a disorder of the respiratory system characterized by inflammation, narrowing of the airways and increased reactivity of the airways to inhaled agents. Initiators of asthma include but are not limited to, allergens, cold temperatures, exercise, viral infections, SO₂.

In one example, the subject has established inflammation.

In one example, the subject does not have cancer.

The subject may be wild-type for the ezh2 gene or mutant for the ezh2 gene. In one example, the mutant is a Y641 mutant of the EZH2 polypeptide. In another example, the mutant has a mutation selected from the group consisting of Y641F, Y641H, Y641 N, and Y641S.

In another aspect, the present disclosure provides an Ezh2 inhibitor as described herein for use in the prevention, reduction in one or more symptoms of inflammation or treatment of inflammation in a subject. In a particular example, the inflammation is allergic inflammation.

In another aspect, the present disclosure provides an Ezh2 inhibitor for use in the prevention, reduction in one or more symptoms of inflammation or treatment of inflammation in a subject. In a particular example, the inflammation is allergic inflammation.

In another example, the present disclosure provides for use of an Ezh2 inhibitor as described herein in the manufacture of a medicament for preventing, reducing one or more symptoms of inflammation or treating inflammation. In a particular example, the inflammation is allergic inflammation.

### BRIEF DESCRIPTION OF THE FIGURES

FIG. 1. protein sequence of human Ezh2.

FIG. 2. Components of the PRC2 and Suv39h-HP1 pathways are specifically up regulated after T cell activation.

a) Quantitation of the expression changes of the genes encoding 34 repressive chromatin components associated with histone modification from publicly available human CD4⁺ T cell microarray data (naïve vs 24 hrs activation with anti-CD3 and anti-CD28) (Martinez-Llordella et al., 2013). Genes significantly up-regulated are denoted in red, genes significantly down-regulated are denoted in blue, and genes not significantly altered are denoted in black. Other genes are denoted in gray. b) Representative western blots of naïve vs 24 hr activated (anti-CD3 and anti-CD28) C57BL/6 mouse CD4⁺ T cells (biological replicates shown) with quantification (mean±SEM from 4 samples) shown in c). Statistical significance was determined by student's t-test. d) Schematic displaying the molecules involved in the H3K27me3-associated PRC2 and H3K9me3 associated Suv39h-HP1 gene silencing.

FIG. 3. Cd4-driven cre recombinase causes efficient deletion of loxP-flanked target gene products.

a) Fluorescence-activated cell sorting of splenic B cells and CD4+ T cells. b) Western blot analysis from 2 representative Cbx5^{fl/fl} Cd4^{Cre}, Cbx1^{fl/fl} Cd4^{Cre} and Trim28^{fl/fl} Cd4^{Cre} mice showing efficient deletion of Cbx5 (HP1α), Cbx1 (HP1β) and Trim28 (TIF1β) respectively in the T cell compartment.

FIG. 4. Suv39h-HP1 family members are not required for development of allergic inflammation.

a) Histological analysis of the airways of Cbx5^{fl/fl} Cd4^{Cre}, Cbx1^{fl/fl} Cd4^{Cre} and Trim28^{fl/fl} Cd4^{Cre} and control floxed mice showing the frequencies of PAS+ cells and tissue inflammation following OVA-induced allergic inflammation. Un-exposed wildtype sections are shown for comparative purposes. Scale bar represents 100 μm. b) Quantification of histological analysis shown in a. Individual data points as well as Mean and SD are shown. Statistical significance was determined by Kruskal-Wallis test with Dunn's post-hoc comparing each floxed control group to un-exposed Bl/6 controls (*P<0.05) and corresponding Cd4Cre mice. Group sizes are n=5 Bl/6, n=9 Cbx5fl/fl and Cbx5fl/flCd4Cre, n=6 Cbx1fl/fl, Cbx1fl/flCd4Cre, Trim28fl/fl and Trim28fl/flCd4Cre. c) Representative flow cytometry analysis of bronchoalveolar lavage samples of indicated genotypes following OVA-induced allergic inflammation. d) Quantification of individual cell populations from flow cytometry shown in c. Eosinophils (CD11 b+, Siglec-F+, CD11c−), Neutrophils (CD11bHi, GR1Hi), CD4+ T Cells (TCRβ+, CD4+, CD8−), CD8+ T Cells (TCRβ+, CD4−, CD8+). Individual data points as well as Mean and SD are shown. Statistical significance was determined as in b. Group sizes are n=4 Bl/6, n=6 all other groups. e) Bio-Plex® analysis of cytokine levels in BAL fluid in mice (group sizes as in b). Only cytokines induced more than 2-fold by OVA are shown. Mean and SEM are shown. Statistical analysis by Kruskal-Wallis test with Dunn's post-hoc.

FIG. 5. Baseline effects of T-cell HP1α, HP1β or TIF1β deletion on lung inflammation levels in bronchoalveolar lavage samples.

Quantification of individual cell populations identified by flow cytometry are shown as mean and SD for n=3 per group. Live cells (Sytox Blue-) were further gated into individual subsets as follows: Eosinophils (CD11b+, Siglec-F+, CD11c−); Alveolar Macrophages (CD11b+, Siglec-F+, CD11c+); Neutrophils (CD11bHi, GR1Hi); B Cells (CD11b−, TCRβ−, CD19+); CD4 T Cells (TCRβ+, CD4+, CD8−); CD8 T Cells (TCRβ+, CD4−, CD8+). a) HP1α deletion and floxed controls. b) HP1β deletion and floxed controls. c) TIF1β deletion and floxed controls.

FIG. 6. Ezh2 is essential for T cells to drive allergic inflammation.

a) Experimental protocol of ovalbumin (OVA)-induced allergic inflammation. b) Representative forward-scatter versus side-scatter flow cytometric plots of bronchoalveolar lavage (BAL) samples from Ezh2^{fl/fl} Cd4^{Cre} and wildtype C57Bl/6 (Bl/6) mice following OVA-induced allergic inflammation. Data shown representative of n=2 (WT/Alum), n=8 (Ezh2^{fl/fl} Cd4^{Cre}/Alum), n=12 (WT/Ova), n=11 (Ezh2^{fl/fl} Cd4^{Cre}/Ova). c) Gating strategy identifying individual leukocyte populations in BAL samples from OVA sensitized and challenged Ezh2^{fl/fl} control and Ezh2^{fl/fl} Cd4^{Cre} animals which are quantified in (d). Mean and SEM for n=4 Bl/6, n=6 Ezh2^{fl/fl}/OVA, n=6 Ezh2^{fl/fl} Cd4^{Cre}/OVA. OVA-sensitized Ezh2^{fl/fl} and Ezh2^{fl/fl} Cd4^{Cre} groups were compared by Mann-Whitney U-test. Un-challenged Bl/6 is shown for comparative purposes. e) Representative histological analysis of the airways of Ezh2^{fl/fl} CD4^{Cre} and Bl/6 mice following OVA-induced allergic inflammation. Scale bar represents 100 μm. f) Quantification of PAS⁺ cells and Inflammation Score. OVA refers to initial sensitization with all mice exposed to nebulized OVA challenge. Mean and SEM shown for n=12 (Bl/6/Ova), n=4 (Ezh2^{fl/fl}/Ova), n=13 (Ezh2^{fl/fl} Cd4^{Cre}/Ova), n=7 (Ezh2^{fl/fl} Cd4^{Cre}/Alum). Statistical significance was determined by Kruskal-Wallis H test with Dunn's post-hoc test. g) Airways resistance (Rn) following OVA-induced allergic inflammation in Ezh2^{fl/fl} (n=7) and Ezh2^{fl/fl} Cd4^{Cre} mice (n=7). Un-challenged Bl/6 mice (n=6) were included to indicate baseline bronchoconstriction to methacholine (MCh). Mean and SEM are shown and data was analysed by 2-way ANOVA with Bonferroni post-hoc test. *P<0.05 with specific comparisons denoted in central and right hand panels.

FIG. 7. Representative forward-scatter versus side-scatter flow cytometric plots of bronchoalveolar lavage (BAL) samples after gating individual leukocyte populations. Gating strategy can be seen in FIG. 5c. Neutrophils are the SSCMid population gated by CD11bHiGR1Hi; Eosinophils are the SSCHi population gated by CD11b+CD11c−SiglecF+; Alveolar macrophages are the FSCHiSSCHi population gated by CD11b+CD11c+SiglecF+; T lymphocytes are the FSCLoSSCLo population gated by CD11b−GR1−TCRβ+.

FIG. 8. Ezh2 deletion in T cell compartment protects against the development of HDM-induced allergic inflammation

a) Experimental protocol of house dust mite (HDM)-induced allergic inflammation b) Flow cytometric analysis of bronchoalveolar lavage (BAL) fluid in Ezh2^{fl/fl} Cd4^{Cre} and control Ezh2^{fl/fl} mice following house dust mite (HDM)-induced allergic inflammation (representative of n=7-8 per group pooled from 3 independent experiments). c) Quantification of total BAL cells (from b.) and individual leukocyte populations. Mean and SEM is shown for n=8 (Ezh2^{fl/fl}/PBS and Ezh2^{fl/fl} Cd4^{Cre}/HDM groups), n=7 (Ezh2^{fl/fl}/HDM and Ezh2^{fl/fl} Cd4^{Cre}/PBS groups). Statistical analysis by 2-way ANOVA with Tukey's multiple comparisons test. d) Representative histological analysis of the airways of Ezh2^{fl/fl} Cd4^{Cre} and wildtype mice following HDM-induced allergic inflammation. Scale bar represents 100 μm. e) Quantification of histological analysis shown in d. Mean and SEM is shown. Group sizes as in c. Statistical analysis by 2-way ANOVA with Tukey's multiple comparisons test.

FIG. 9. Expression of PRC2 core components are critical for the development of allergic inflammation.

a) Histological analysis of the airways of Eed^{fl/fl} Cd4^{Cre}, Suz12^{fl/fl} Cd4^{Cre} and C57Bl/6 (Bl/6) wildtype mice following OVA challenge. Representative samples are shown on the left panels. The frequency of PAS+ cells and the Inflammation Score is shown on the right as Mean and SEM. Group sizes are n=12 Eed^{fl/fl} Cd4^{Cre} with n=11 age-matched Bl/6 controls, n=5 Suz12^{fl/fl} Cd4^{Cre} with n=5 age-matched Bl/6 controls. Statistical significance was determined by Mann-Whitney U-test. b) Representative flow cytometric analysis of bronchoalveolar lavage (BAL) infiltrate in Suz12^{fl/fl}Cd4^{Cre} and wildtype Bl/6 mice following OVA-induced asthma. Populations corresponding to granulocytes, lymphocytes and alveolar macrophages are indicated. The frequency of eosinophils (CD11b+, SiglecF+) in the granulocyte gate is depicted on the right. c) Grouped data from b. Mean and SEM shown for n=3 each group. Statistical analysis by 2-way ANOVA with Tukey's post-hoc. d) Bio-Plex® analysis of cytokine levels in BAL fluid in Eed^{fl/fl} Cd4^{Cre} (n=10), Suz12^{fl/fl} Cd4^{Cre} (n=3) and wildtype Bl/6 (n=5) mice following OVA-induced asthma. Only cytokines induced more than 2-fold by OVA are shown (mean and SEM) and statistical significance was determined by two-way ANOVA.

FIG. 10. Ezh2 is required to generate antigen-specific memory.

a) Bio-Plex® analysis of cytokine levels in bronchoalveolar lavage (BAL) fluid following OVA challenge in Ezh2^{fl/fl} Cd4^{Cre} and control Ezh2 full mice. Mean fold change over level in control alum-sensitized mice is shown with associated SD. Statistical analysis by multiple unpaired t-tests corrected for multiple comparisons using the Holm-Sidak method n=2 (Ezh2^{fl/fl}), n=8 (Ezh2^{fl/fl} Cd4^{Cre}). Only cytokines induced more than 2-fold by OVA are shown. b) IFNγ levels in BAL fluid following OVA challenge. X-axis labels indicate initial sensitization. Mean fold change over control and SD is shown. Statistical analysis by 2-way ANOVA. Group sizes as in a. c) OVA-specific IgE and total IgE detected in serum of mice following OVA challenge. X-axis labels indicate initial sensitization. Individual data points as well as Mean and SD are shown, n=3 each group except for un-immunized Bl/6 where n=1 is shown for reference. Statistical analysis by Kruskal-Wallis H test with Dunn's post-hoc. d) Intracellular cytokine staining of splenic antigen-experienced (CD44⁺) CD4⁺ T cells 3 days after OVA sensitization protocol (10 days after initial sensitization). Cells were stimulated with PMA/Ionomycin stimulation for 5 h in the presence of Golgistop protein transport inhibitor for the final 2 h. e) Quantification of d. Mean and SEM is shown. Statistical analysis by Mann-Whitney U-test with n=6 per group. f) OVA class II tetramer staining of cells pooled from mechanically homogenized spleen and peripheral lymph nodes after OVA sensitization protocol as in d. g) Quantification of f. Mean and SEM is shown for n=3 (Bl/6), n=6 (Ezh2^{fl/fl}), n=5 (Ezh2^{fl/fl} Cd4^{Cre}). Statistical analysis by Kruskal-Wallis H test with Dunn's post-hoc.

FIG. 11. Ezh2 is required for CD4+ T cell clonal expansion

a) Proliferation of cell-trace violet labelled naïve CD4+ T cells from Ezh2^{fl/fl} Cd4^{Cre} and wildtype following anti-CD3, anti-CD28 activation in vitro. Up-regulation of the activation marker CD25 is displayed in the bottom panel. b) Cell numbers recovered from Ezh2^{fl/fl} Cd4^{Cre} and wildtype cultures from a. Mean and SEM is shown for n=3. Statistical analysis by 2-way ANOVA with Bonferroni post-hoc. c) Annexin-V and propidium iodide staining of cells activated as in a. Live cells (bottom left quadrant), early apoptotic cells (top left quadrant) and late apoptotic/necrotic cells (top right quadrant). d) Quantification of c. Group sizes and statistical analysis as in b. *P<0.05, **P<0.01, ***P<0.001 compared to Bl/6 control at that time-point, or as indicated.

FIG. 12. Small molecule inhibition of Ezh2 suppresses the development of allergic inflammation.

a) Effect of Ezh2 inhibitor GSK126 on survival of activated C57BL/6 mouse CD4⁺ T cells after 3 days in vitro. Data are shown as the mean % of the vehicle control cell number ±SEM from 3-5 measurements at each concentration of GSK126. b) Experimental protocol of GSK126 administration by oral gavage in ovalbumin (OVA)-induced allergic inflammation. c) Quantification of flow cytometry analysis of bronchoalveolar lavage infiltrate following GSK126 administration in OVA model of allergic inflammation in C57BL/6 mice (n=4 per group). Mean and SEM is shown. Statistical analysis by Kruskal-Wallis H test with Dunn's post-hoc. d) Representative histological analysis (representative of n=8 per group, pooled from 2 independent experiments) of the airways of mice treated with GSK126 or vehicle control in OVA model as per b. Scale bar represents 100 μm. e) Quantification of histological analysis showing the frequency of PAS⁺ cells and the Inflammation Score. Mean and SEM are shown for n=8 per group. Statistical significance was determined by Student's t-test. f) OVA-specific IgE and total IgE detected in serum of mice following OVA challenge and GSK126 administration. Mean and SEM are shown for n=5 (Vehicle), n=4 (each GSK dose). Statistical analysis by Kruskal-Wallis H test with Dunn's post-hoc. g) Airways resistance (Rn) following OVA-induced allergic inflammation in C57BL/6 mice treated with vehicle or GSK126. Full methacholine dose-response for Vehicle and GSK126 (150 mg/kg) treated groups is shown in left panel and all groups airways resistance to 10 mg/mL methacholine is shown in right panel. Mean and SEM are shown for n=6 (Saline/Vehicle), n=6 (OVA/Vehicle), n=5 (OVA/75 mg/kg GSK126), n=5 (OVA/150 mg/kg GSK126) and data was analysed by 2-way ANOVA with Bonferroni post-hoc test. **P<0.01 with specific comparisons denoted in right-hand panel.

FIG. 13. Splenic cell numbers and proportions are unchanged following 4 days oral administration of the Ezh2 inhibitor GSK126

Flow cytometry analysis of splenic cell proportions in C57BL/6 mice following administration of GSK126 (75 mg/kg and 150 mg/kg) or vehicle control in OVA-induced asthma model (experimental protocol as per FIG. 5b). a) Representative plots. b) Quantification of total splenocytes and individual leukocyte populations. Mean and SEM as well as individual data points are shown. n=4 each group. Data was statistically tested by 1-way ANOVA with Dunnet's post-hoc test.

FIG. 14. A single dose of GSK126 selectively reduces CD4+ T cell numbers in the airways

a) Experimental protocol using one dose of Ezh2 inhibitor GSK126 (150 mg/kg) in established inflammation. b) Bronchoalveolar lavage (BAL) cell numbers following single dose GSK126 or vehicle in established inflammation. CD4+ and CD8+ T cells were first gated by CD11b−GR1−TCRβ+. Neutrophils were gated by CD11bHiGR1Hi. Eosinophils were gated by CD11b+CD11c−SiglecF+. Mean and SEM are shown. n=2 Alum-sensitized vehicle treated controls shown for comparative purposes. OVA-sensitized groups were statistically compared by Student's t test. Group sizes were n=4 Vehicle and n=5 GSK126.

FIG. 15. Inhibition of Ezh2 can treat established allergic inflammation.

a) Experimental protocol using Ezh2 inhibitor GSK126 in established inflammation. b) Representative flow cytometry plots of bronchoalveolar lavage (BAL) samples from GSK126 (150 mg/kg by oral gavage) and vehicle treated C57Bl/6 mice. Data shown representative of n=5 OVA/Vehicle (Veh) and OVA/GSK groups, n=2 unchallenged controls (Con) shown for reference purposes. c) Quantification of b. Mean and SEM shown. Group sizes as in b. Statistical analysis by Mann-Whitney U-test comparing Vehicle and GSK126 groups. d) Correlation of BAL eosinophils with BAL CD4⁺ T cells in groups as per b. Individual data points shown. e) Representative histological analysis of the airways of mice following GSK126 or vehicle treatment in established inflammation. Scale bar represents 100 μm. f) Quantification of PAS⁺ cells and Inflammation Score. Mean and SEM shown. Group sizes as in b. Statistical analysis by 1-way ANOVA with Holm-Sidak post-hoc.

FIG. 16. Histological scoring chart to quantify leukocyte infiltration in H&E stained lung sections.

### DETAILED DESCRIPTION OF THE DISCLOSURE

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, Perbal (1984), Sambrook et al., (1989), Brown (1991), Glover and Hames (1995 and 1996), and Ausubel et al., (1988, including all updates until present), Harlow and Lane, (1988), Coligan et al., (including all updates until present) and Zola (1987).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

The present invention employs conventional molecule biology, microbiology and recombinant DNA techniques within the skill of the art. See for example, Sambrook et al “Molecular Cloning” A Laboratory Manual (1989).

The terms “consisting of” or “consisting essentially of” refers to a peptide sequence of a defined number of residues which is not covalently attached to a larger product.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example herein shall be taken to apply mutatis mutandis to any other example unless specifically stated otherwise

### Selected Definitions

As used herein, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent (%), up or down (higher or lower).

The term “allergic inflammation” as used herein is intended to refer to a disorder which is triggered by expose to an allergen such as a pollen, dust mite, mould, animal skin/hair.

The term “asthma” as used herein refers to a lung disease characterized by airway obstruction that is reversible (although not entirely in some patients) either spontaneously or with treatment, airway inflammation, and increased airway responsiveness to a variety of stimuli. Asthma may be broadly categorised into allergic asthma and non-allergic (intrinsic) asthma. “Allergic asthma” as used herein refers to an asthmatic response to inhalation of an antigen to which the patient is sensitive. Non-allergic asthma is also known as Th2-low asthma and intrinsic asthma and can include late-onset asthma. Intrinsic asthma may be brought on by one of more of the following, stress, anxiety, cold and/or dry air, cigarette smoke, viruses, air pollution, chemicals, fragrances, strenuous exercise (exercise-induced asthma) and drugs (e.g. aspirin, and non-steroidal inflammatory drugs (NSAIDs).

The term “allergic rhinitis” (commonly known as hay-fever) as used herein is caused by the nose and/or eyes of a subject coming into contact with environmental allergens such as pollens, dust mite, moulds and animal hair/skin. Typically, symptoms include runny nose, rubbing of the nose, itchy nose, sneezing and itchy, watery eyes.

The term “atopic dermatitis” (commonly known as eczema) as used herein refers to an inherited, chronic inflammatory condition in which patches of skin become red, scaly and itchy. Sometimes lesions appears which can become infected. Factors such as contact with irritants in the environment often trigger atopic dermatitis.

The term “autoimmune disease” refers to conditions in which a person's immune system produces an inappropriate response against its own cells, tissues and/or organs. This results in inflammation and damage. The term “autoimmune disease” is intended to encompass local or systemic diseases.

The term “COPD” as used herein refers to the collective term for a number of lung diseases that prevent proper breathing. The two most common types of COPD are emphysema and chronic bronchitis. COPD are breathlessness, chronic cough and sputum (mucus or phlegm) production.

The term “lupus” as used herein refers to a chronic autoimmune disease that can affect any organ. The term “lupus” includes systemic lupus erythematosus (SLE), discoid lupus, subacute cutaneous lupus and drug-induce lupus.

The term “coeliac disease’ refers to an autoimmune disease caused by abnormal response to gluten that damages the small bowel and affects food absorption.

Reference to Ezh2 or EZH2 are used interchangeably to refer to the protein unless the context dictates otherwise. Ezh2 or ezh2 in italics is intended to refer to the gene unless the context dictates otherwise.

The term “pharmaceutical composition”, as used herein, means any composition, which contains at least one therapeutically or biologically active agent and is suitable for administration to the patient. Any of these formulations can be prepared by well-known and accepted methods of the art. See, for example, Gennaro, A. R., ed., Remington: The Science and Practice of Pharmacy, 20th Edition, Mack Publishing Co., Easton, Pa. (2000).

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

By “subject” is meant any subject, particularly a mammalian subject, for whom diagnosis, prognosis, or therapy is desired. As used herein, the term “subject” includes any human or nonhuman animal. The term “nonhuman animal” includes all vertebrates, e.g., mammals and non-mammals, such as nonhuman primates, sheep, dogs, cats, horses, cows, bears, chickens, amphibians, reptiles, etc. and may, where appropriate, be used interchangeably with the term “patient”. Preferably, the subject is a primate. Particularly, the subject is a human.

The term “therapeutically effective amount” shall be taken to mean a sufficient quantity of an Ezh2 inhibitor to realise a desired biological effect. For example, an effective amount of an Ezh2 inhibitor is that amount necessary to reduce or inhibit one or more symptoms of allergic inflammation to a level that is below that observed and accepted as clinically characteristic of that disorder (e.g. by preventing or reducing the development of IgE in response to an allergen. This term is not to be construed to limit the invention to a specific quantity.

As used herein, the terms “treat,” “treating,” “treatment” and grammatical variations thereof mean subjecting an individual patient to a protocol, regimen, process or remedy, in which it is desired to obtain a physiologic response or outcome in that patient. Since every treated patient may not respond to a particular treatment protocol, regimen, process or remedy, treating does not require that the desired physiologic response or outcome be achieved in each and every patient or patient population. Accordingly, a given patient or patient population may fail to respond or respond inadequately to treatment.

As used herein, the term “prevent”, “prevented”, or “preventing” when used with respect to the treatment of an allergic or asthmatic disorder refers to a prophylactic treatment which increases the resistance of a subject to an allergen or initiator or, in other words, decreases the likelihood that the subject will develop an allergic or asthmatic response to the allergen or initiator as well as a treatment after the allergic or asthmatic disorder has begun in order to fight the allergy/asthma, e g reduce or eliminate it altogether or prevent it from becoming worse.

### Allergic Inflammation

The term “allergy” was coined in 1906 to call attention to the unusual propensity of some individuals to develop signs and symptoms of reactivity, or ‘hypersensitivity reactions’, when exposed to certain substances (allergens). Allergens fall into two main categories. The first type encompasses any non-infectious environmental substance that can induce IgE production (thereby ‘sensitizing’ the subject) so that later re-exposure to that substance induces an allergic reaction. Common sources of allergens include grass and tree pollens, animal dander (sheddings from skin and fur), house-dust-mite faecal particles, certain foods (notably peanuts, tree nuts, fish, shellfish, milk and eggs), latex, some medicines and insect venoms. In some instances, allergen-specific IgE directed against foreign antigens can also recognize cross-reactive host antigens, but the clinical significance of this is unclear. The second type is a non-infectious environmental substance that can induce an adaptive immune response associated with local inflammation but is thought to occur independently of IgE (for example, allergic contact dermatitis to poison ivy or nickel).

Allergic disorders are increasingly prevalent in the developed world and include allergic rhinitis (also known as hay fever), atopic dermatitis (also known as eczema), allergic (or atopic) asthma and some food allergies (Holgate S T. (1999) Nature 404:B2-B4). Some people develop a potentially fatal systemic allergic reaction, termed anaphylaxis, within seconds or minutes of exposure to allergens (Sampson H A et al. (2005) J Allergy Clin Immunol. 115:584-591).

Allergic inflammation refers to the inflammation produced in sensitized subjects after expose to a specific allergen(s). A single allergen produces an acute reaction, which is known as an early-phase reaction or a type I immediate hypersensitivity reaction. In many subjects, this is followed by a late-phase reaction. With persistent or repetitive exposure to allergen, chronic allergic inflammation develops, with associated tissue alterations.

In recent years, it has become clear that much of the pathology, and therefore the burden of disease, associated with allergic disorders reflects the long-term consequences of chronic allergic inflammation at sites of persistent or repetitive exposure to allergens. This realization has led to renewed efforts to define additional therapeutic targets in allergic disease, to devise improved strategies to induce immunological tolerance to the offending allergens, and even to manipulate the immune response to prevent the initial development of allergic disorders.

Allergic inflammation often is classified into three temporal phases. Early-phase reactions are induced within seconds to minutes of allergen challenge, and late-phase reactions occur within several hours. By contrast, chronic allergic inflammation is a persistent inflammation that occurs at sites of repeated allergen exposure.

Early-phase reactions (or type I immediate hypersensitivity reactions) occur within minutes of allergen exposure and mainly reflect the secretion of mediators by mast cells at the affected site. Reactions can be localized (for example, acute rhinoconjunctivitis in allergic rhinitis, acute asthma attacks, urticaria (hives) and gastrointestinal reactions in food allergies) or systemic (anaphylaxis). In sensitized individuals, these mast cells already have allergen-specific IgE bound to their surface high-affinity IgE receptors (FcεRI). When crosslinking of adjacent IgE molecules by bivalent or multivalent allergen occurs, aggregation of FcεRI triggers a complex intracellular signalling process that results in the secretion of three classes of biologically active product: those stored in the cytoplasmic granules, lipid-derived mediators, and newly synthesized cytokines, chemokines and growth factors, as well as other products. These events cause vasodilation, increased vascular permeability with oedema, and acute functional changes in affected organs (such as bronchoconstriction, airway mucus secretion, urticaria, vomiting and diarrhoea). Some of the released mediators also promote the local recruitment and activation of leukocytes, contributing to the development of late-phase reactions.

A late phase reaction refers to a reaction that typically develops after 2-6 h and peaks 6-9 h after allergen exposure. It is usually preceded by a clinically evident early-phase reaction and fully resolves in 1-2 days. Skin late-phase reactions involve oedema, pain, warmth and erythema (redness). In the lungs, these reactions are characterized by airway narrowing and mucus hypersecretion. They reflect the local recruitment and activation of TH2 cells, eosinophils, basophils and other leukocytes, and persistent mediator production by resident cells (such as mast cells). Mediators that initiate late-phase reactions are thought to be derived from resident mast cells activated by IgE and allergen or from T cells that recognize allergen-derived peptides (such T cells may be either resident at, or recruited to, sites of allergen challenge). Certain mast-cell-derived products can also influence the biology of structural cells, including vascular endothelial cells, epithelial cells, fibroblasts, smooth muscle cells and nerve cells. Other products that contribute to late-phase reactions can be derived from T cells that recognize allergen-derived peptides; such T cells may be either resident at or recruited to early-phase reactions at sites of allergen challenge.

Chronic allergic inflammation refers to persistent inflammation induced by prolonged or repetitive exposure to specific allergens, typically characterized not only by the presence of large numbers of innate and adaptive immune cells (in the form of leukocytes) at the affected site but also by substantial changes in the extracellular matrix and alterations in the number, phenotype and function of structural cells in the affected tissues.

Many patients who initially have a single allergic disorder, such as atopic dermatitis, eventually develop others, such as allergic rhinitis and allergic asthma (this is called the allergic march or atopic march). This process may be driven in part by a vicious circle in which allergic inflammation diminishes the function of the epithelial barrier. This increases the immune system's exposure to the original allergens and additional allergens, and existing allergen-specific IgE contributes to sensitization to new allergens. In this scheme, antigen-presenting cells (APCs) that express surface FceRI and/or the low-affinity IgE receptor CD23 (including FceRI-bearing Langerhans cells and other dendritic cells, as well as CD23-bearing B cells) capture allergens by means of their surface-bound allergen-specific IgE. By processing these IgE-bound antigens, APCs can promote the development of TH2-cell responses to other epitopes of the allergen for which sensitization already exists or to other allergens that are being processed in parallel by the same APCs. This proposed mechanism may result in epitope spreading (the production of IgE specific for multiple epitopes on single allergens and IgE specific for new allergens).

### Zeste Homolog 2 (EZH2) and Inhibitors Thereof

The post-translational modification of histones plays a key role in the regulation of gene expression and cellular differentiation (Strahl B D, Allis C D. (2000) Nature. 403:41-45). Enhancer of zeste homolog 2 (EZH2) is a pleiotropically acting molecule; its primary conserved function is in epigenetic gene suppression as an essential component of polycomb repressive complex 2 (PRC2).

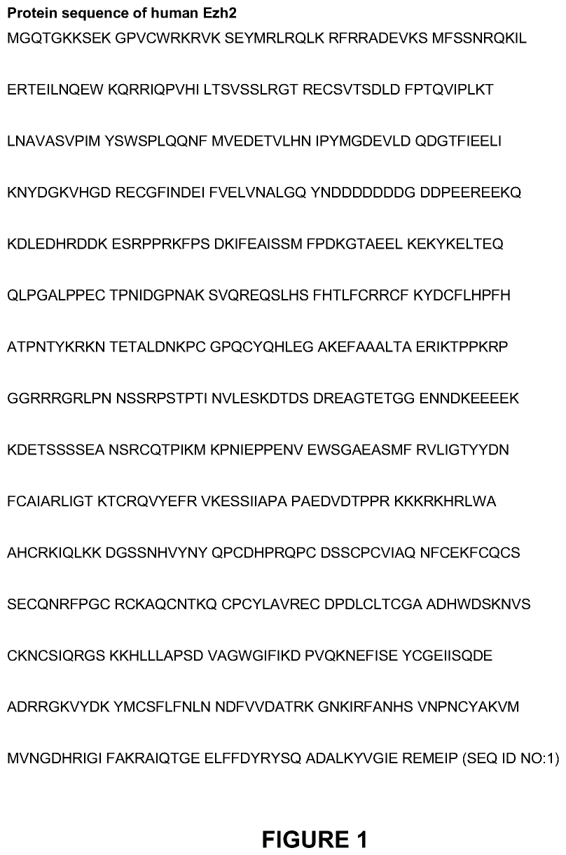

The sequence of the human Ezh2 protein sequence can be found at UniProt reference Q15910. This sequence is also reproduced herein in FIG. 1 (SEQ ID NO:1). The term Ezh2 inhibitor is also intended to include its isoforms.

EZH2 catalyzes the methylation of the ε-NH2 group of histone 3 lysine 27 (H3K27) in the nucleosome substrate, via transfer of a methyl group from the cofactor S—(S′-adenosyl)-I-methionine (SAM), leading to trimethylation of H3K27 (H3K27me3) and transcriptional silencing of target genes. Accumulated evidence suggests that EZH2 is deeply involved in aberrant transcriptome in cancer cells. Indeed, EZH2 and the product of its enzymatic action H3K27me3 have been associated with poor prognosis in a variety of human malignancies. EZH2 amplification and functional alteration are frequently observed in a majority of cancers.

Alterations in EZH2 or the SWI/SNF complex, which antagonizes polycomb function, have been described in multiple cancer subtypes including breast cancer, ovarian cancer, prostate cancer, non-Hodgkin lymphoma (NHL), and T-cell ALL (McCabe et al. (2012) Nature 492:108). Recurrent gain-of-function alterations in EZH2 occur in up to 30% of germinal center B-cell like diffuse large B-cell lymphoma (GCB-DLBCL) and 27% of follicular lymphoma (FL).

Interestingly, a study in 2013 implicated Ezh2 in allergic asthma development as Ezh2-deficient T cells were shown to enhance asthma pathology after adoptive transfer of in vitro-activated T cells (Tumes et al. (2013) Immunity 39:819-832). The studies presented herein clearly contrast with those findings. Tumes et al. used a Th2 in vitro polarization of OVA-specific T cell receptor transgenic Ezh2 deficient CD4+ T cells prior to adoptive transfer into OVA sensitized mice. In contrast, here the inventors directly tested the importance of Ezh2 to the generation of T cell responses in vivo.

Small molecule inhibitors of EZH2 have recently been developed and are currently being evaluated in clinical trials. Several small molecules that suppress the enzymatic activity of EZH2 have been recently developed. While most compounds are still in preclinical development, three agents (tazemetostat, GSK2816126 and CPI-1205) have moved into phase I/II clinical trials (summarised in Gulati N et al. (2018) Leukemia and Lymphoma 59(7):1574-1585) and a number of active clinical trials in lymphoma with these agents is currently undergoing.

### EPZ-6438 (E7438/Tazemetostat)

Tazemetostat (from Epizyme, Inc) is an orally bioavailable small molecule inhibitor of EZH2 (Knutson S K et al. (2013) Proc Natl Acad Sci USA. May 7; 110(19):7922-7). Tazemetostat was optimized from EPZ 005687 (Knutson S K et al. (2012) Nt Chem Biol 8(11):890-6), a molecule identified in 2012 by a high throughput screen of a chemical diversity library against PRC2. EPZ 005687 demonstrates high affinity and selectivity for EZH2, however has suboptimal pharmacokinetic properties that limit its clinical utility. Tazemetostat has increased potency and improved pharmacokinetics including oral bioavailability (Knutson S K et al. (2013) supra). Tazemetostat inhibits EZH2 through competitive inhibition with the cofactor S-adenosyl-L-methionine (SAM), which is required for EZH2 function. Tazemetostat inhibits both wild type and mutant forms of EZH2 with a 50% inhibitory concentration (IC50) ranging from 2-38 nM. Tazemetostat is also highly selective for EZH2 with 35-fold increased potency relative to EZH1 and >4,500-fold increased potency relative to 14 other histone methyl-transferases (Knutson et al. (2013) supra).

The preclinical activity of tazemetostat has been evaluated in NHL, multiple myeloma (MM) and select solid tumors with dependency on PRC2 function (Knutson S K, et al. (2014) Mol Cancer Ther. 2014; 13:842-854).

In addition, tazemetostat has been studied in solid tumors with notable preclinical activity in pediatric malignant rhabdoid tumors, which harbor inactivating biallelic mutations in the SWI/SNF subunit SMARCB1 as well as SMARCB1-deficient synovial sarcoma.

The encouraging preclinical studies of tazemetostat led to the clinical development of this compound which is now being studied in a series of phase I and phase II trials in NHL and genetically defined solid tumors. Preliminary results from a large phase I/II trial of tazemetostat (NCT01897571) have recently been reported (Morschhauser F, et al. (2017) Hematol Oncol. 2017; 35:24-25). Other ongoing studies evaluating tazemetostat in patients with NHL include a phase Ib/II trial of tazemetostat in combination with R-CHOP (rituximab, cyclophosphamide, doxorubicin, vincristine, prednisone) in patients with previously untreated DLBCL, a phase I trial of tazemetostat in combination with the PD-1 inhibitor atezolimab in patients with relapsed/refractory DLBCL or FL, and a phase II trial in pediatric patients with lymphoma or genetically defined solid tumors.

The United States Food and Drug Administration has granted tazemetostat a Fast Track designation for EZH2 mutant DLBCL and for FL regardless of EZH2 mutation status as well as an Orphan Drug designation for malignant rhabdoid tumors.

### GSK2816126 (GSK126)

GSK126 (GlaxoSmithKline) is a small molecular inhibitor of EZH2, which was generated through chemical optimization of a compound identified from a high throughput biochemical screen of compounds targeting EZH2 (McCabe M T et al. (2012) Nature December 6; 492(7427):108-12). Similar to EPZ-6438, GSK126 inhibits both WT and mutant EZH2 through competitive inhibition with S-adenosylmethionine (SAM). The predicted docking site of GSK126 is the SAM binding pocket of EZH2. GSK126 inhibits WT and mutant EZH2 with similar potency (Ki app¼0.5-3 nM) and is highly selective when compared to EZH1 (150-fold increased potency) or 20 other methyltransferases (>1000-fold increased potency) (McCabe eta. supra).

GSK126 was recently evaluated in a multicenter phase I clinical trial (NCT 02082977).

GSK have also developed further EZH2 inhibitors, GSK343 (Verma S et al. (2012) ACS Med Chem Lett 13; 3(12):1091-1096) and GSK503 (Bequelin W et al. (2013) 13; 23(5):677-92).

### CPI-1205

Constellation Pharmaceuticals has reported a series of indole-based EZH2 inhibitors which are structurally unique from the pyridine based compounds GSK126 and tazemetostat (Gehling V S, et al. (2015) Bioorg Med Chem Lett. 2015; 25.3644-3649). CPI-1205 is an orally bioavailable, indole-based, small molecule inhibitor of EZH2. It is a N-trifluoroethylpiperidine analog of the chemical probe CPI-169 (Gehling et al. supra). CPI-169 demonstrated antitumor activity and PD target engagement in-vivo, however it had limited oral bioavailability. CPI-1205 binds to the EZH2 catalytic pocket and partially overlaps with the SAM binding site. It has shown modest selectivity for EZH2 over EZH1 (EZH1 IC50 52±11 nm) and selectivity when tested against 30 other histone or DNA methyltransferases (Vaswani R G, et al. J Med Chem. 2016; 59:9928-9941).

CPI-1205 is currently being evaluated in a phase I clinical trial in patients with relapsed or refractory B-cell lymphoma (NCT 02395601). This study opened in March 2015 and is expected to accrue approximately 41 patients through October 2018. The primary objective is to describe the dose-limiting toxicities of CPI-1205. PK, PD, and response assessment will also be studied.

### EI1

Novartis have developed an S-Adenosyl methionine (SAM) competitive inhibitor of Ezh2, EI1, which inhibits the methyltransferase activity of the Ezh2/PRC2. EI1-treated cells exhibit genome-wide loss of H3K27 methylation and activation of PRC2 target genes. Furthermore, inhibition of Ezh2 by EI1 in diffused large B-cell lymphomas cells carrying the Y641 mutations resulted in decreased proliferation, cell cycle arrest, and apoptosis (Wei Qi et al. (2012) PNAS 109(52):21360-21365).

### EZH2 Inhibitors in Preclinical Development

With an increasing understanding of the role that EZH2 plays in oncogenesis and encouraging data from clinical trials, additional EZH2 inhibitors continue to be synthesized and evaluated in the preclinical setting (Stazi G, et al. (2014-2016). Expert Opin Therapeu Patents. 2017; 27:797-813). Many of the most potent EZH2 inhibitors have a pyridone moiety, however several groups have patented alternative EZH2 inhibitors and are testing the effect of replacement or addition of different substituents to the 2-pyridone moiety on the efficacy and potency of the drug. Examples of such patents describing mutant EZH2 inhibitors include (WO 2012/034132, WO 2018/133795, US2013/0040906, US2011/0251216, US2009/0012031, US2018/0271892, US 2018/0243315, US2017/0065600, US2015/0141362, U.S. Pat. Nos. 9,949,999, 9,889,180, 9,889,138, and 9,688,665,).

Another strategy to optimize efficacy has been to develop agents that target both EZH2 and EZH1 simultaneously. For example, the dual EZH1/EZH2 inhibitor UNC 1999 has been demonstrated to inhibit growth of MLL-rearranged leukemia in-vitro and in vivo. In addition, the recently described dual EZH1/EZH2 inhibitors OR-S1 and OR-S2 demonstrated greater antitumor activity than selective EZH2 inhibitors, both in-vitro and in-vivo against DLBCL cells harbouring gain of function mutations in EZH2 (Honma D et al. (2017) Cancer Sci. 108:2069-2078). In total, >50 small molecular inhibitors of EZH2 are currently in various stages of pre-clinical development.

Other inhibitors of EZH2 have been described in the literature, including DZNep (Fiskus W et al. (2009) 24; 114(13):2733-43), UNC199 (Konze K D et al. (2013) ACS Chem Biol 8(6):1324-34) and SAH-EZH2 (Kim W et al. (2013) 9(10):643-50).

The EZH2 inhibitors are available from a number of different commercial suppliers including Cayman Chemical, Sigma (Merck), SelleckChem, AdooQ Bioscience, and APExBio.

TABLE 1
Commercially available EzH2 inhibitors

| Product | Company | Details |
|---|---|---|
| GSK126 | Cayman Chemical | Item No. 15415 |
| | | CAS Number 1346574-57-9 |
| | | C₃₁H₃₈N₆O₂ |
| GSK343 | Merck | Catalog No. SML 0766 Sigma |
| | | CAS Number 1346704-33-3 |
| | | C₃₁H₃₉N₇O₂ |
| GSK503 | Cayman Chemical | Item No. 18531 |
| | | CAS Number 1346572-63-1 |
| | | C₃₁H₃₈N₆O₂ |
| EPZ005687 | Cayman Chemical | Item No. 13966 |
| | | CAS Number 1396772-26-1 |
| | | C₃₂H₃₇N₅O₃ |
| EPZ-6438 | SelleckChem | Catalog No. S7128 |
| | | CAS Number 1403254-99-8 |
| | | C₃₄H₄₄N₄O₄ |
| EI1 | Cayman Chemical | Item No. 19146 |
| | | CAS Number 1418308-27-6 |
| | | C₂₃H₂₆N₄O₂ |
| CPI-1205 | KareBay Biochem | Catalog No. KI6172 |
| | | CAS Number 1621862-70-1 |
| | | C₂₇H₃₃F₃N₄O³ |

Prophylaxis and/or Treatment of Allergic Inflammation

In some examples, the Ezh2 inhibitor is used in prophylaxis of allergic inflammation in a subject at risk of developing an allergy (e.g. asthma) where the expose of the subject to an allergen or predisposition to asthma is known to be suspected. A “subject at risk” as described herein is a subject who has any risk of exposure to an allergen or a risk of developing an allergic inflammatory condition (e.g. asthma) and/or someone who has suffered from an asthmatic attack previously or has a predisposition to asthmatic attacks. For instance, a subject at risk may be a subject who is planning to travel to an area where a particular type of allergen is found or prevalent or it may even be any subject living in an area where an allergen has been identified. If the subject develops allergic responses to a particular antigen and the subject may be exposed to the antigen, i.e., during pollen season, then that subject is at risk of exposure to the antigen. A subject at risk of developing an allergic inflammation (e.g. asthma) includes those subjects that have been identified as having an allergy but that don't have the active disease during the treatment of the method of the disclosure as well as subjects that are considered to be at risk of developing these diseases because of genetic or environmental factors.

In other examples, the Ezh2 inhibitor is used in the treatment of allergic inflammation in a subject having an allergy. A “subject having an allergy” is a subject that has an allergic reaction in response to an allergen. An “allergy” refers to acquired hypersensitivity to a substance (allergen). The allergic reaction in humans and animals has been extensively studied and the basic immune mechanisms involved are well known. Allergic conditions or diseases in humans include but are not limited to eczema, allergic rhinitis or coryza, hay fever, conjunctivitis, bronchial or allergic asthma, urticaria (hives) and food allergies; atopic dermatitis; anaphylaxis; drug allergy; angioedema; and allergic conjunctivitis.

The generic name for molecules that cause an allergic reaction is allergen. There are numerous species of allergens. The allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens. Histamine, platelet activating factor, arachidonic acid metabolites, and serotonin are among the best known mediators of allergic reactions in human subjects. Histamine and the other vasoactive amines are normally stored in mast cells and basophil leukocytes. The mast cells are dispersed throughout tissue and the basophils circulate within the vascular system. These cells manufacture and store histamine within the cell unless the specialized sequence of events involving IgE binding occurs to trigger its release.

Delayed type hypersensitivity, also known as type IV allergy reaction is an allergic reaction characterized by a delay period of at least 12 hours from invasion of the antigen into the allergic subject until appearance of the inflammatory or immune reaction. The T lymphocytes (sensitized T lymphocytes) of individuals in an allergic condition react with the antigen, triggering the T lymphocytes to release lymphokines (macrophage migration inhibitory factor (MIF), macrophage activating factor (MAF), mitogenic factor (MF), skin-reactive factor (SRF), chemotactic factor, neovascularization-accelerating factor, etc.), which function as inflammation mediators, and the biological activity of these lymphokines, together with the direct and indirect effects of locally appearing lymphocytes and other inflammatory immune cells, give rise to the type IV allergy reaction. Delayed allergy reactions include tuberculin type reaction, homograft rejection reaction, cell-dependent type protective reaction, contact dermatitis hypersensitivity reaction, and the like, which are known to be most strongly suppressed by steroidal agents. Consequently, steroidal agents are effective against diseases which are caused by delayed allergy reactions. Long-term use of steroidal agents at concentrations currently being used can, however, lead to the serious side-effect known as steroid dependence. The methods of the disclosure solve some of these problems, by providing for lower and fewer doses to be administered.

Immediate hypersensitivity (or anaphylactic response) is a form of allergic reaction which develops very quickly, i.e. within seconds or minutes of exposure of the patient to the causative allergen, and it is mediated by IgE antibodies made by B lymphocytes. In non-allergic patients, there is no IgE antibody of clinical relevance; but, in a person suffering with allergic diseases, IgE antibody mediates immediate hypersensitivity by sensitizing mast cells which are abundant in the skin, lymphoid organs, in the membranes of the eye, nose and mouth, and in the respiratory tract and intestines.

Mast cells have surface receptors for IgE, and the IgE antibodies in allergy-suffering patients become bound to them. As discussed briefly above, when the bound IgE is subsequently contacted by the appropriate allergen, the mast cell is caused to degranulate and to release various substances called bioactive mediators, such as histamine, into the surrounding tissue. It is the biologic activity of these substances which is responsible for the clinical symptoms typical of immediate hypersensitivity; namely, contraction of smooth muscle in the airways or the intestine, the dilation of small blood vessels and the increase in their permeability to water and plasma proteins, the secretion of thick sticky mucus, and in the skin, redness, swelling and the stimulation of nerve endings that results in itching or pain.

Many allergies are caused by IgE antibody generation against harmless allergens. The cytokines that are induced by administration of immunostimulatory nucleic acids are predominantly of a class called “Th1” (examples are IL-12 and IFN-γ). Cytokine production by helper CD4+(and also in CD8+) T cells frequently fall into one of two phenotypes, Th1 and Th2, in both murine and human systems (Romagnani, (1991) Immunol Today 12: 256-257, Mosmann, (1989) Annu Rev Immunol, 7: 145-173). Th1 cells produce interleukin 2 (IL-2), tumour necrosis factor (TNFα) and interferon gamma (IFNγ) and they are responsible primarily for cell-mediated immunity such as delayed type hypersensitivity. Th2 cells produce interleukins, IL-4, IL-5, IL-6, IL-9, IL-10 and IL-13 and are primarily involved in providing optimal help for humoral immune responses such as IgE and IgG4 antibody isotype switching (Mosmann, (1989), Annu Rev Immunol, 7: 145-173).

The types of antibodies associated with a Th1 response are generally more protective because they have high neutralization and opsonization capabilities. Th2 responses involve predominately antibodies and these have less protective effect against infection and some Th2 isotypes (e.g., IgE) are associated with allergy.

Strongly polarized Th1 and Th2 responses not only play different roles in protection, they can promote different immunopathological reactions. Th1-type responses are involved in organ specific autoimmunity such as experimental autoimmune uveoretinitis (Dubey et al, 1991, Eur Cytokine Network 2: 147-152), experimental autoimmune encephalitis (EAE) (Beraud et al, 1991, Cell Immunol 133: 379-389) and insulin dependent diabetes mellitus (Hahn et al, 1987, Eur. J. Immunol. 18: 2037-2042), in contact dermatitis (Kapsenberg et al, Immunol Today 12: 392-395), and in some chronic inflammatory disorders. In contrast Th2-type responses are responsible for triggering allergic atopic disorders (against common environmental allergens) such as allergic asthma (Walker et al, 1992, Am Rev Resp Dis 148: 109-115) and atopic dermatitis (van der Heijden et al, 1991, J Invest Derm 97: 389-394), are thought to exacerbate infection with tissue-dwelling protozoa such as helminths (Finkelman et al, 1991, Immunoparasitol Today 12: A62-66) and Leishmania major (Caceres-Dittmar et al, 1993, Clin Exp Immunol 91: 500-505), are preferentially induced in certain primary immunodeficiencies such as hyper-IgE syndrome (Del Prete et al, 1989, J Clin Invest 84: 1830-1835) and Omenn's syndrome (Schandene et al, 1993, Eur J Immunol 23: 56-60), and are associated with reduced ability to suppress HIV replication (Barker et al, 1995, Proc Soc Nat Acad Sci USA 92: 11135-11139).

Thus, in general, allergic diseases are mediated by Th2 type immune responses.

An “allergen” as used herein is a molecule capable of provoking an immune response characterized by production of IgE. Thus, in the context of this disclosure, the term allergen means a specific type of antigen which can trigger an allergic response which can be mediated by IgE antibody.

The allergens that may trigger an allergic inflammation of the disclosure may cover a broad class, including fragments of such allergens or haptens acting as allergens. Allergens include but are not limited to Environmental Aeroallergens; plant pollens such as Ragweed/hayfever; Weed pollen allergens; Grass pollen allergens; Johnson grass; Tree pollen allergens; Ryegrass; House dust mite allergens; Storage mite allergens; Japanese cedar pollen/hay fever Mold spore allergens; Animal allergens (cat, dog, guinea pig, hamster, gerbil, rat, mouse); Food Allergens (e.g., Crustaceans; nuts, such as peanuts; citrus fruits); Insect Allergens (Other than mites listed above); Venoms: (Hymenoptera, yellow jacket, honey bee, wasp, hornet, fire ant); Other environmental insect allergens from cockroaches, fleas, mosquitoes, etc.: Bacteria such as streptococcal antigens; Parasites such as Ascaris antigen; Viral Antigens; Fungal spores; Drug Allergens; Antibiotics; penicillins and related compounds; other antibiotics; Whole Proteins such as hormones (insulin), enzymes (Streptokinase); all drugs and their metabolites capable of acting as incomplete antigens or haptens; Industrial Chemicals and metabolites capable of acting as haptens and stimulating the immune system (Examples are the acid anhydrides (such as trimellitic anhydride) and the isocyanates (such as toluene diisocyanate)); Occupational Allergens such as flour (ie. Baker's asthma), castor bean, coffee bean, and industrial chemicals described above; flea allergens; and human proteins in non-human animals.

Allergens include but are not limited to cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many allergens, however, are protein or polypeptide in nature, as proteins and polypeptides are generally more antigenic than carbohydrates or fats.

Examples of specific natural, animal and plant allergens include but are not limited to proteins specific to the following genuses: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

In another example, the present disclosure provides methods for preventing, reducing or treating allergic inflammation in a hypo-responsive subject. As used herein, a hypo-responsive subject is one who has previously failed to respond to a treatment directed at treating or preventing asthma or allergy or one who is at risk of not responding to such a treatment. Subjects who are hypo-responsive include those who are refractory to an asthma/allergy medicament. As used herein, the term “refractory” means resistant or failure to yield to treatment. Such subjects may be those who never responded to an asthma/allergy medicament (i.e., subjects who are non-responders), or alternatively, they may be those who at one time responded to an asthma/allergy medicament, but have since that time have become refractory to the medicament. In some embodiments, the subject is one who is refractory to a subset of medicaments. A subset of medicaments is at least one medicament. In some embodiments, a subset refers to 2, 3, 4, 5, 6, 7, 8, 9, or 10 medicaments. In one example, a hypo-responsive subjects is an elderly subject, regardless of whether they have or have not previously responded to a treatment directed at treating or preventing allergic inflammation (e.g. asthma). In another example, the hypo-responsive subject is a neonatal subject.

Administration of the Ezh2 inhibitor to the subject may be by any suitable means, for example intravenous, oral, transdermal, systemic and/or intramuscular route. In some examples, the Ezh2 inhibitor is administered by inhalation. In some examples, the Ezh2 inhibitor may be co-administered with a drug which may be an β-adrenergic agent, a theophylline compound, a corticosteroid, an anticholinergic, an antihistamine, a calcium channel blocker, a cromolyn sodium, or a combination thereof.

In some examples, the Ezh2 inhibitor is co-administered with an allergy medicament. Allergy medicaments include, but are not limited to, anti-histamines, steroids, and prostaglandin inducers. Anti-histamines are compounds which counteract histamine released by mast cells or basophils. These compounds are well known in the art and commonly used for the treatment of allergy. Anti-histamines include, but are not limited to, loratidine, cetirizine, buclizine, ceterizine analogues, fexofenadine, terfenadine, desloratadine, norastemizole, epinastine, ebastine, ebastine, astemizole, levocabastine, azelastine, tranilast, terfenadine, mizolastine, betatastine, CS 560, and HSR 609. Prostaglandin inducers are compounds which induce prostaglandin activity. Prostaglandins function by regulating smooth muscle relaxation. Prostaglandin inducers include, but are not limited to, S-5751.

In some examples, the Ezh2 inhibitor may be administered in combination with a steroid, immune-modulator or non-steroidal glucocorticoid receptor agonist. Steroids include, but are not limited to, beclomethasone, fluticasone, triamcinolone, budesonide, corticosteroids and budesonide. Corticosteroids are used long-term to prevent development of the symptoms, and suppress, control, and reverse inflammation arising from an initiator. Some corticosteroids can be administered by inhalation and others are administered systemically. The corticosteroids that are inhaled have an anti-inflammatory function by blocking late-reaction allergen and reducing airway hyper-responsiveness. These drugs also inhibit cytokine production, adhesion protein activation, and inflammatory cell migration and activation.

Corticosteroids include, but are not limited to, beclomethasome dipropionate, budesonide, flunisolide, fluticaosone, propionate, and triamcinoone acetonide. Although dexamethasone is a corticosteroid having anti-inflammatory action, it is not regularly used for the treatment of asthma/allergy in an inhaled form because it is highly absorbed, it has long-term suppressive side effects at an effective dose. Systemic corticosteroids include, but are not limited to, methylprednisolone, prednisolone and prednisone. Cortosteroids are used generally for moderate to severe exacerbations to prevent the progression, reverse inflammation and speed recovery. These anti-inflammatory compounds include, but are not limited to, methylprednisolone, prednisolone, and prednisone.

Immunomodulators include, but are not limited to, the group consisting of anti-inflammatory agents, leukotriene antagonists, IL-4 muteins, soluble IL-4 receptors. immunosuppressants (such as tolerizing peptide vaccine), anti-IL-4 antibodies, IL-4 antagonists, anti-IL-4Ra antibodies, anti-IL-5 antibodies, anti-IL-5 receptor antibodies, soluble IL-13 receptor-Fc fusion proteins, anti-IL-9 antibodies, CCR3 antagonists, CCR5 antagonists, VLA-4 inhibitors, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets.

Other immunomodulators include neuropeptides that have been shown to have immunomodulating properties. Functional studies have shown that substance P, for instance, can influence lymphocyte function by specific receptor mediated mechanisms. Substance P also has been shown to modulate distinct immediate hypersensitivity responses by stimulating the generation of arachidonic acid-derived mediators from mucosal mast cells. J. McGillies, et al., Substance P and Immunoregulation, Fed. Proc. 46:196-9 (1987).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment.

Long-term control medications include compounds such as corticosteroids (also referred to as glucocorticoids), methylprednisolone, prednisolone, prednisone, cromolyn sodium, nedocromil, long-acting 132-agonists, methylxanthines, muscarinic receptor antagonists and leukotriene modifiers. Quick relief medications are useful for providing quick relief of symptoms arising from allergic or asthmatic responses. Quick relief medications include short-acting 132 agonists, anticholinergics and systemic corticosteroids.

Cromolyn sodium and nedocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from eosinophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratropium bromide. These compounds reverse only cholinerigically-mediated bronchospasm and do not modify any reaction to antigen.

Prophylaxis and/or Treatment of Lymphocyte-Induced Inflammation

In certain examples, the Ezh2 inhibitor is used in the prophylaxis of a subject either at risk of developing lymphocyte-induced inflammation or in the treatment of a subject who has a lymphocyte-induced inflammation. A lymphocyte-induced inflammation refers to any inflammatory condition which is mediated by the immune system, most notably cells of the T lineage such as T-helper 1 and Th17 cells. The role of these cells in inflammation has been extensively published (see for example Crane I J et al. (2005) Crit. Rev. Immunol. 25(2):75-102). Chemokines associated with a Th1-type inflammatory reaction include IFN-γ, CXCL10, CXCL9, CXCL11, CCL3, CCL4, and CCL5 and these contribute to the high pathogenic potential at the sites of inflammation. The main route to chemokine production in inflamed tissue is via inflammatory cytokines, which via a Th1 response include IL-1, TNF-α and IFN-γ.

The general perception in the art is that human autoimmune diseases such as multiple sclerosis (MS), type-1 insulin-dependent diabetes mellitus, Hashimoto's thyroiditis and rheumatoid arthritis are Th1-mediated diseases. The methods described herein are suitable for the treatment of any autoimmune disease which is Th1 mediated including, but not limited to lupus (e.g. systemic lupus erythematosus), coeliac disease, acute disseminated encephalomyelitis, acute motor axonal neuropathy, Addison's disease, adiposis dolorosa (Dercum's disease), adult onset still's disease, alopecia areata, ankylosing spondylitis, antiphospholipid syndrome, antisynthetase syndrome, autoimmune pancreatitis, autoimmune thrombocytopenic purpura, autoimmune urticaria, Balo concentric sclerosis, Bechet's disease, Bickerstaff encephalitis, bullous pemphigoid, coeliac disease, chronic fatigue syndrome, chronic inflammatory demyelinating polyneuropathy, cicatricial pemphigoid, Crohn's disease, dermatomyositis, diabetes mellitus type 1, endometriosis fibromyalgia, gastritis, giant cell arteritis, Graves' disease, Graves ophtalmopathy, Guillain barre syndrome, Hashimoto encephalopathy, Hashimoto thyroiditis, juvenile arthritis, lichen planus, Lyme Disease, multiple sclerosis, myasthenia gravis, myocarditis, neuromyelitis optica, pediatric acute-onset, neuropsychiatric syndrome, postmyocardial infarction syndrome, psoriasis, restless leg syndrome, rheumatic fever, systemic lupus erythematosus, scleroderma, Sjogren's, transverse myelitis, ulcerative colitis, vasculitis and vitiligo.

### Prophylaxis and Treatment of Non-Allergic Asthma

The methods of the present disclosure also encompass the prevention, reduction and/or treatment of non-allergic asthma. In non-allergic or intrinsic asthma, allergens have no obvious role in driving the inflammatory process in the airways. Symptoms may be provoked by bacterial infections or virus infections associated with sinusitis or bronchitis. Symptoms may also be provoked by other non-allergic factors including weather changes, exercise, indoor pollutants, outdoor pollutants, strong odours or chemicals.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive. The present disclosure includes the following non-limiting examples.

### EXAMPLES

### Methods

### Mice

Ezh2^{fl/fl} (Su et al., (2003) Nat Immunol 4, 124-131) Eed^{fl/fl} (Xie et al. (2014) Cell Stem Cell 14, 68-80), Suz12^{fl/fl} (Lee et al. (2015) Blood 126, 167-175), Cbx5^{fl/fl} (Allan et al. (2012) Nature 487, 249-253) and Trim28^{fl/fl} (Cammas et al. (2000) Development 127, 2955-2963) mice were described previously.

Cbx1^{fl/fl} mice were a kind gift from Dr Florence Cammas (IGBMC, Strasbourg). The floxed strains were crossed to Cd4Cre mice (Lee et al. (2001) Immunity 15, 763-774). All mice lines were maintained on a C57BL/6 (Ly5.2) background and were used between 6 and 12 weeks of age and were age and sex matched. Animal experiments were conducted in accordance with the guidelines of the Walter and Eliza Hall Institute (WEHI) Animal Ethics Committee.

### Ovalbumin Model of Allergic Lung Inflammation

Mice were immunized with an intraperitoneal injection of 200 μL Alum/OVA containing 20 μg low-endotoxin ovalbumin (Worthington) and 2.25 mg aluminium hydroxide (Sigma) in sterile PBS on day 0 and day 7. Mice were then rested for a minimum of 2 weeks and then challenged daily for 4 days with nebulized 2% (w/v) ovalbumin (Sigma) in PBS for 15 min. The day after the final challenge (or at a time-point described in the text), mice were sacrificed and bronchoalveolar lavage (250 μL×2 with sterile PBS) was performed from which cellular infiltrate was analysed by flow cytometry and acellular BAL fluid was analysed by Bio-Plex Pro™ cytokine assay (Biorad). Lung tissue was then fixed by inserting 1 mL of 10% formalin via the trachea into the lungs leaving to set in situ for at least 30 min. The mid-section of the left lobe was then excised and placed in liquid formalin for a further 24 h prior to paraffin embedding, sectioning and staining with Periodic acid-Schiff (PAS) and Haematoxylin and Eosin (H&E) stains. Stained slides were imaged using an Aperio Digital Pathology Slide Scanner and quantitated by counting PAS positive epithelial cells and scoring leukocyte infiltration in H&E stained sections according to the scoring chart (FIG. 16). Scores from at least five 20× magnification fields of each stain were averaged for each mouse.

### Respiratory Mechanics

Lung function was assessed by the forced oscillation technique using a FlexiVent system with FX1 module (Scireq, Montreal, Canada). Mice were anaesthetized with ketamine (150 mg/kg) and xylazine (15 mg/kg) then cannulated by tracheostomy with ligation. Baseline respiratory mechanics were recorded, followed by aerosolized saline then increasing doses of methacholine (MCh) (0.1-30 mg/mL). Respiratory impedance (Zᵣₛ) was measured and partitioned into airway and parenchymal components through fitting to the Constant Phase Model from which Newtonian resistance (Rₙ; equivalent to airway resistance) was calculated.

### House Dust Mite Model of Allergic Asthma

Mice were challenged with intranasal administration of protein extract from whole house dust mites (HDM; D. Pteronyssinus, Greer Laboratories Inc.) on days 0-2 (10 μg/mouse) and then on days 14-17 (1 μg/mouse) and then sacrificed on day 18. Endpoints were the same as for the ovalbumin model above.

### GSK126 In Vivo Administration

GSK126 (Xcessbio biosciences) was administered by oral gavage at doses of 75 mg/kg and 150 mg/kg each day of exposure to nebulized ovalbumin (4 hours after Ova challenge). GSK126 was dissolved in 20% Captisol® diluent for in vivo administration by oral gavage.

### Enzyme-Linked Immunosorbent Assays (ELISA)

Total IgE and OVA-specific IgE were measured by enzyme-linked immunosorbent assay (ELISA). Briefly, 96 well plates were coated with either Rat anti-mouse IgE (to measure total IgE; Southern Biotech clone #23G2, cat #1130-01) or OVA protein (for OVA-specific IgE) in PBS overnight. Samples were diluted (total IgE 1:50-1:200 and OVA-specific IgE 1:50-1:100 based on pilot assays), and incubated in duplicate, prior to incubation with GAM Fc-specific polyclonal anti-IgE-HRP and colorimetric detection using TMB substrate (Thermo Scientific #34028), measuring absorbance at 450 nm. Absorbance values were converted to total IgE levels by reference to a mouse IgE anti-DNP (clone SPE-7) standard curve (0.1 ng/mL-1 μg/mL) which was included on the same plate. OVA-specific IgE absorbance was expressed relative to a reference OVA-immunized WT mouse which was selected prior to measurement. All ELISAs were performed blinded to treatment and genotype.

### T Cell Cultures

Nave CD4⁺ T cells were isolated from a single-cell suspension from mouse spleen by MACS isolation kit (Miltenyi Biotech). Cells were labelled with CellTrace™ Violet (CTV, Thermo Fisher) then activated in 96 well plates pre-coated with anti-CD3 (10 μg/mL) and anti-CD28 (5 μg/mL) in RPMI-1640 medium supplemented with 10% heat-inactivated fetal bovine serum, 2 mM GlutaMAX™ and 0.05 mM β-mercaptoethanol. Where indicated, cells were treated with the Ezh2 inhibitor GSK126 at the onset of cell activation.

### Immunoblotting

For immunoblotting whole cell extracts were prepared by lysing cells in RIPA buffer (Millipore). Bradford assay was performed to quantify total protein content in each lysate and samples of equivalent protein content were resolved in denaturing conditions in 4-12% gradient SDS-PAGE (Life technologies) and were transferred onto nitrocellulose membrane (Biorad). Membranes were probed with the following antibodies: goat anti-actin-HRP (sc-1616, Santa Cruz), rabbit anti-Ezh2 (#12408, Cell Signaling), rabbit anti-Suz12 (Cell Signaling), mouse anti-HP1a (#05-689, Millipore), mouse anti-HP1p (#MAB3448, Millipore), rabbit anti-LaminB1 (#ab16048, Abcam) mouse anti-TIF1β (#MAB3662, Millipore).

### Flow Cytometry

The following staining panels of fluorochrome-conjugated antibodies against mouse antigens were used for analysis and sorting by flow cytometry.
1. Lavage and spleen immunophenotyping panel: CD19 (1D3)-BUV395 and SiglecF (E50-2440)-PE from BD Pharmingen; CD11c(N418)-FITC, CD8α (53-6.7)-PerCPe710, Ly6c (HK1.4)-e450, GR1(RB6-8C5)-PECy7 and TCRβ(H57-597)-APCe780 from eBioscience; CD4(GK1.5)-AlexaFluor647 and CD11b(M1/70)-AlexaFluor700 were generated internally.
2. Intracellular cytokine analysis: IL-4(11B11)-BV421, CD4(GK1.5)-PECy7, CD44(IM7)-APCCy7 from Biolegend; IFNγ(XMG1.2)-APC, TCRβ(H57-597)-PE from BD Pharmingen; B220(RA3-6B2)-FITC was generated internally.
3. OVA tetramer panel: CD4(GK1.5)-PECy7 and CD19(6D5)-PerCPCy5.5 or B220(RA3-6B2)-Pacific Blue from Biolegend; CD44(IM7)-FITC from BD Pharmingen; TCRβ(H57-597)-APCe780 from eBioscience; CD11b(M1/70)-AlexaFluor700 was generated internally.
4. T cell activation panel: CD25(PC61.5)-PerCPCy5.5 from eBioscience, CD69(H1.2F3)-APC from Miltenyi Biotech.
5. CD4+ T cell and B cell sorting panel: TCRβ(H57-597)-APC from eBioscience; CD4(GK1.5)-PE, CD8α(53-6.7)-FITC, CD19(1D3)-Pacific Blue were generated internally.

Surface staining was carried out at 4° C. for 30 mins and propidium iodide or SytoxBlue exclusion was used as live cell indicator.

Intracellular cytokine staining was performed using FoxP3/Transcription Factor Staining Buffer Set (Affymetrix eBioscience #00-5523) following manufacturer's instructions. PMA (20 ng/mL) and lonomycin (500 ng/mL) stimulation for 5 h was used to stimulate cytokine production and BD GolgiStop™ Protein Transport Inhibitor (BD Pharmingen #554724) was added for final 2 h to allow intracellular accumulation.

Tetramer-based enrichment of OVA-specific CD4+ T cells was performed using PE-conjugated OVA-2C and OVA-3C Peptide:MHC class II tetramers generously provided by Dr James Moon (Massachusetts General Hospital, USA), as described in (Moon et al., 2011). Annexin-V-FITC (BD Pharmingen #556419) staining was performed in 1× binding buffer (10 mM HEPES, 140 mM NaCl, 2.5 mM CaCl₂) in PBS) prior to propidium iodide addition and analysis. In all experiments, stained cells were analysed using BD FACS Canto II, BD FORTESSA X-20 using SPHERO™ Rainbow Calibration Beads to calculate absolute cell counts. Where indicated, cells were sorted using BD FACSARIA III.

### Bioinformatic Analysis

The human naïve CD4⁺ T cell activation microarray dataset originally produced by Martinez-Llordella et al. (2013) J Exp Med 210, 1603-1619) is publically available through the Gene Expression Omnibus (GEO), accession GSE39594. These data were produced using the Affymetrix Human Genome U133 Plus 2.0 Array platform. These data are comprised of CD4+ T cells that have been cultured under different conditions non-stimulated and anti-CD3 plus anti-CD28 for 24 hours. Each group contained 3 replicate samples giving a total of 6 samples. All CEL files were downloaded and probe expression was calculated and normalized using robust multi-array average expression (RMA) (Irizarry et al. (2003) Biostatistics 4, 249-264). Probes were filtered from the data if their expression was less than 6 in at least 3 samples. Probes with no entrez gene ID were also removed. Analysis of these data was performed using the limma (Ritchie et al. (2015) Nucleic acids research 43, e47) software package. Differential expression was evaluated between groups relative to a fold change threshold of 1.2 using linear models and empirical bayes moderated t-statistics with a trended prior-variance (McCarthy and Smyth (2009) Bioinformatics 25, 765-771). P-values were adjusted using the Benjamini and Hochberg method to control the FDR below 5%. The mean-difference plot was produced using limma's plotMD function.

### Example 1 Identification of Heterochromatin Components Up Regulated During CDC T Cell Activation

The inventors investigated whether chromatin-associated proteins which play important regulatory roles in the allergic immune response and as such represent potential therapeutic targets, are up-regulated after T cell activation. To identify such molecules, they examined the expression of genes encoding 34 different repressive chromatin components associated with histone modification in publicly available microarray data comparing non-stimulated human CD4⁺ T cells to those stimulated for 24 hours (Martinez-Llordella et al. (2013) J Exp Med 210, 1603-1619).

Molecules associated with PRC2-H3K27me3 (EZH2, EED, RBBP7) and the Suv39h-HP1-H3K9me3 (SUV39H1, SUV39H2, CBX1, CBX3, CBX5 and TRIM28) pathways were significantly up regulated (adjusted p<0.05) in response to T cell activation whereas other components such as PRC1 members (BMI1, RING1, CBX2, 4, 7 and 8), most HDACs (excluding HDAC2) and other lysine methyltransferases such as DOT1L, EZH1, EHMT1, 2 and SETDB1 were not significantly altered or even down regulated (FIG. 1a). Thus, components of two major epigenetic silencing pathways shown in FIG. 1d were up regulated after T cell activation.

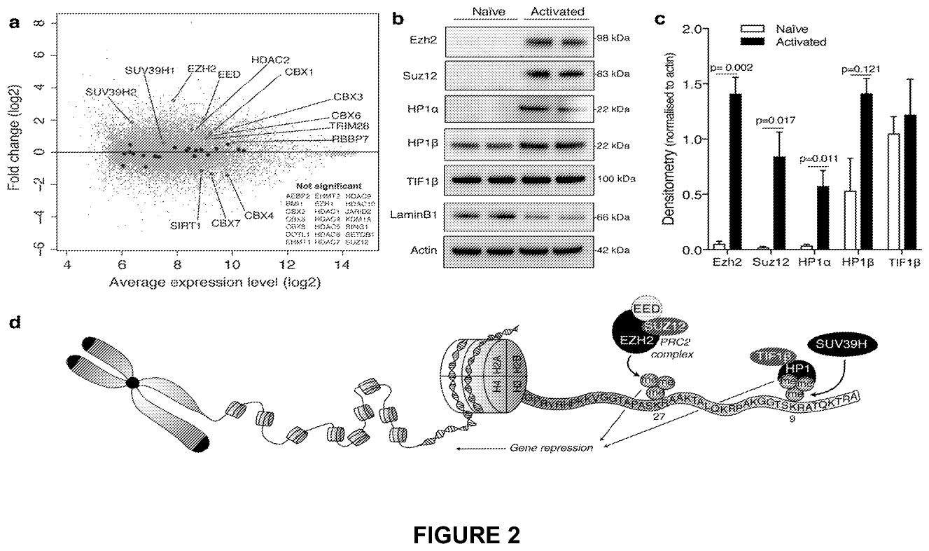

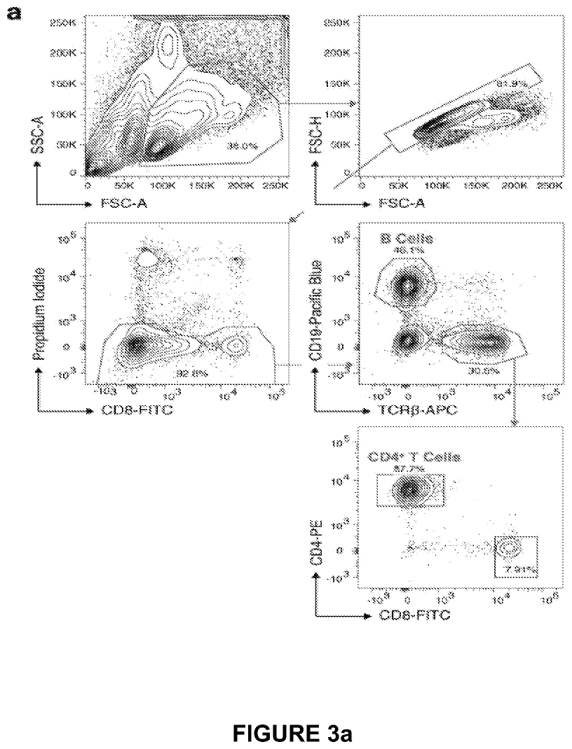

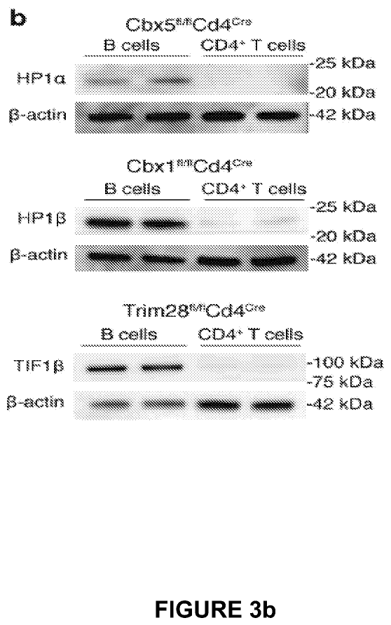

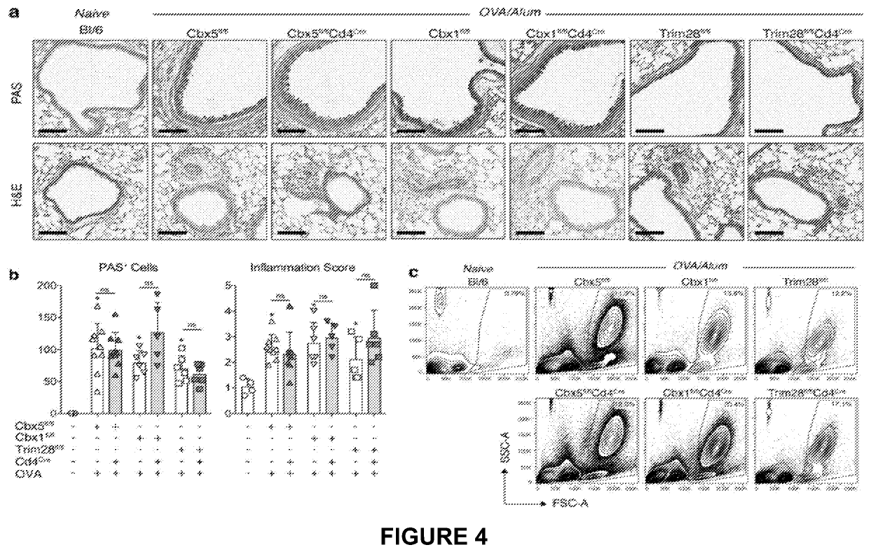

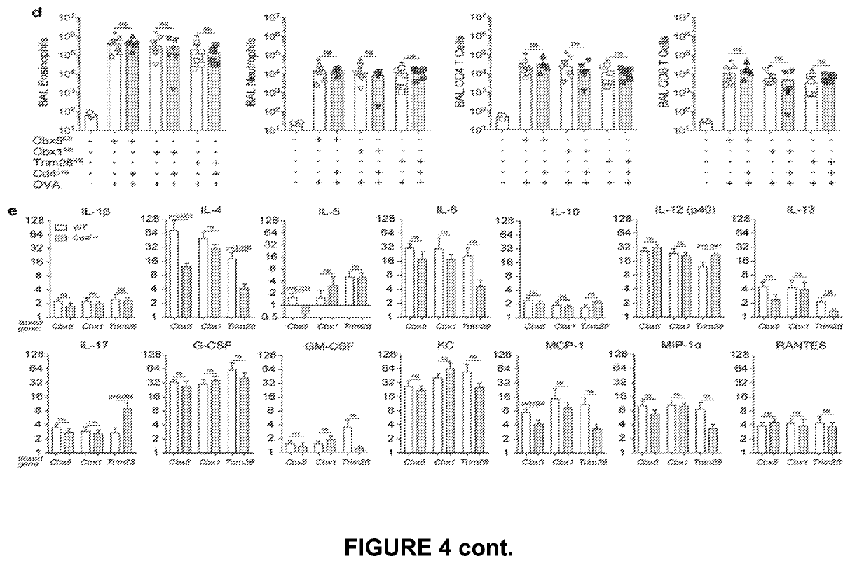

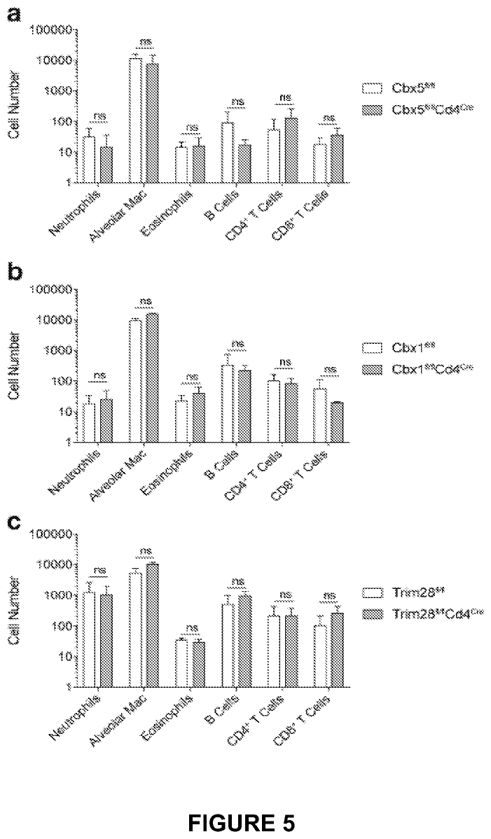

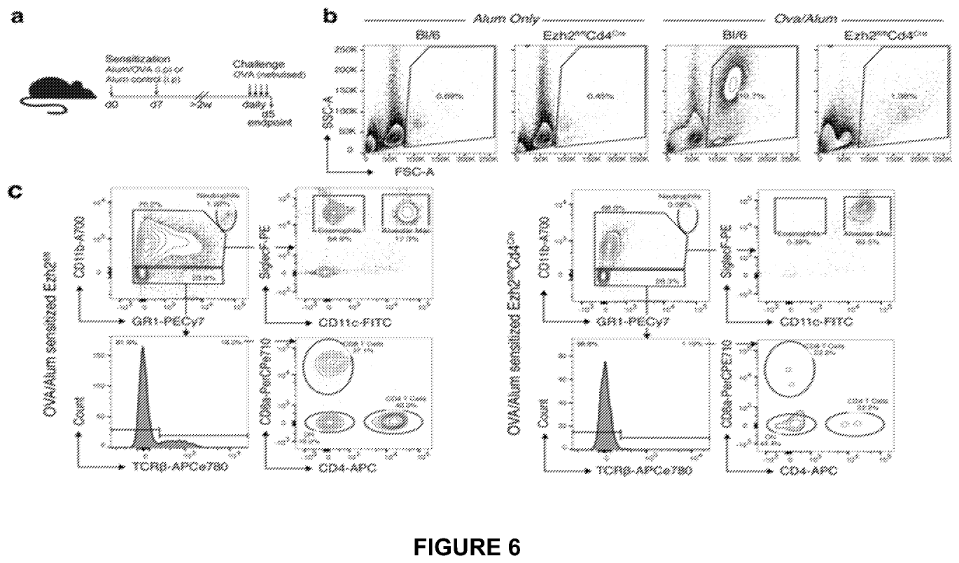

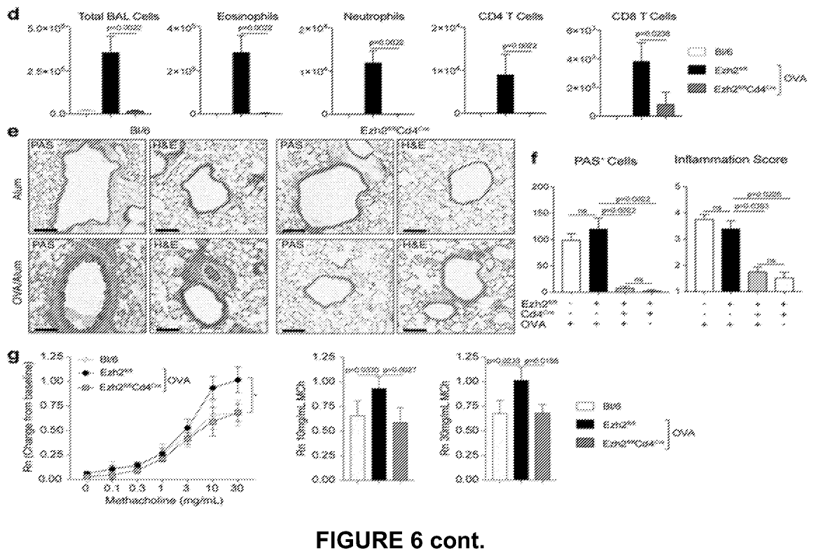

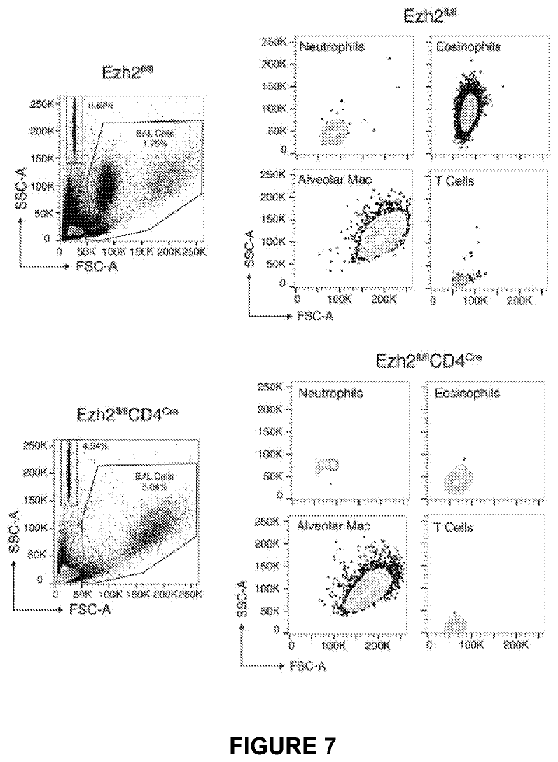

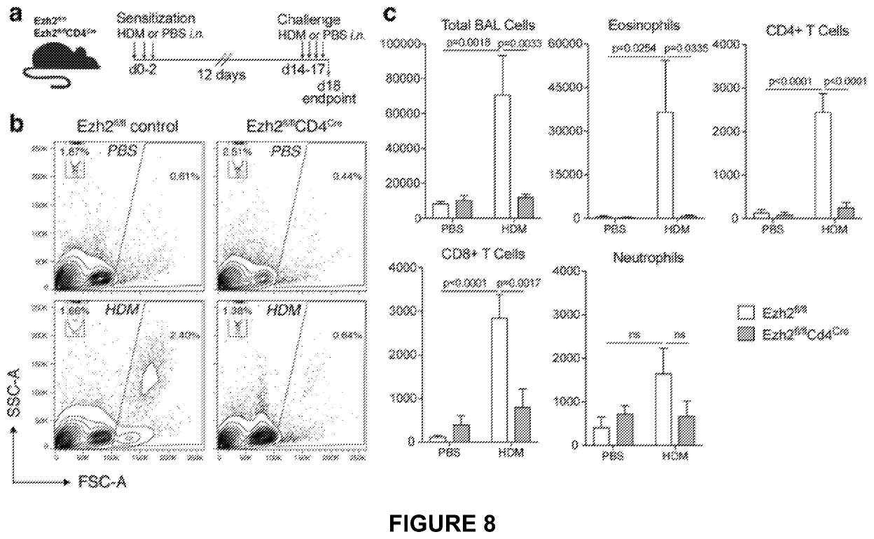

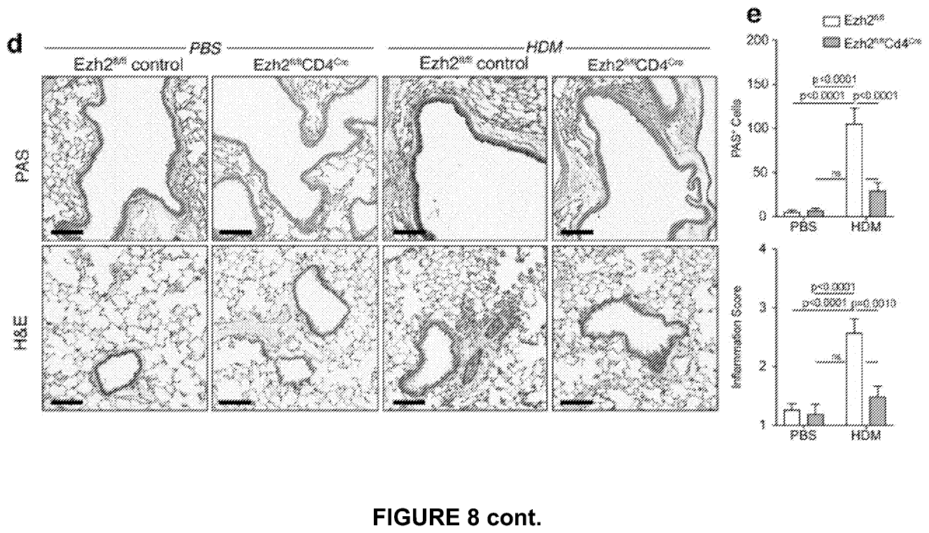

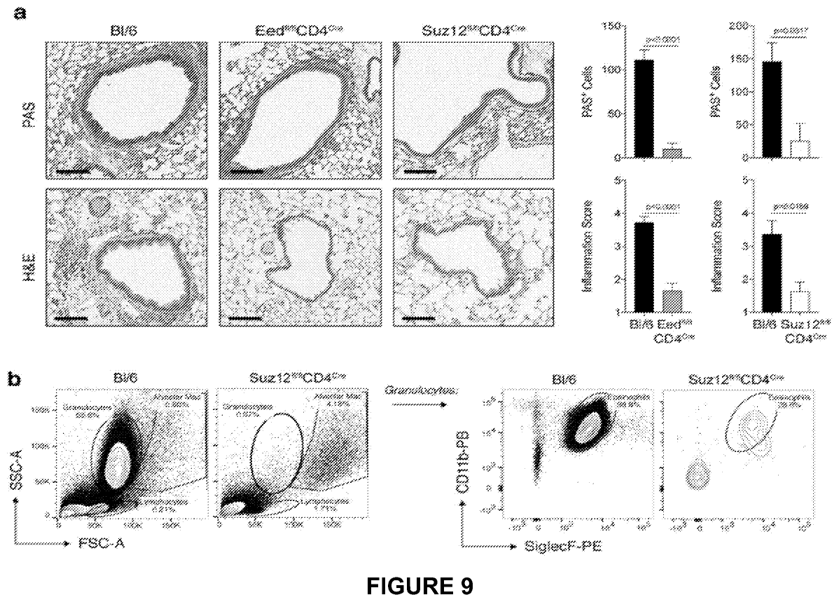

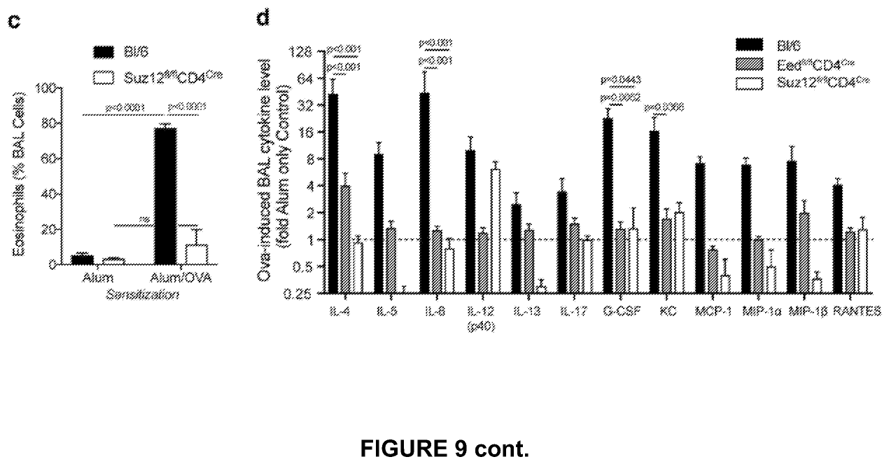

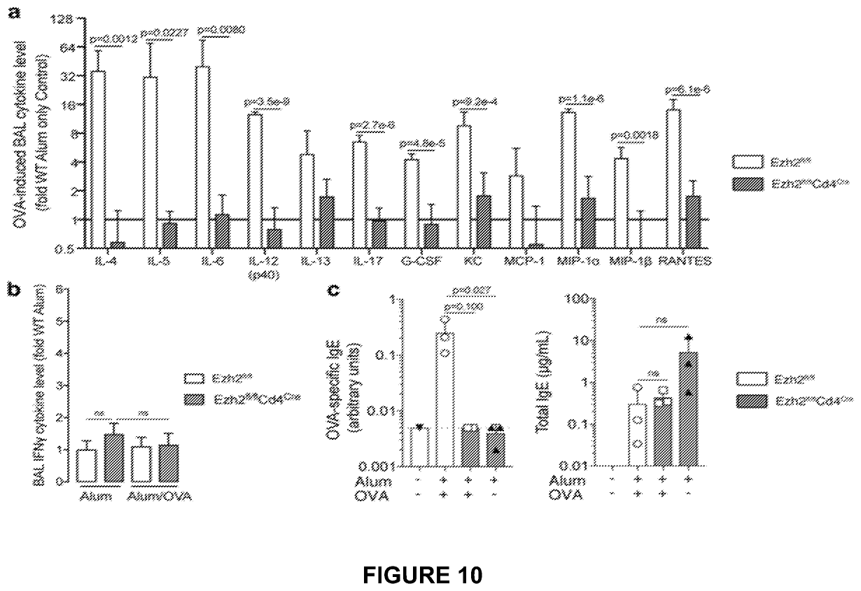

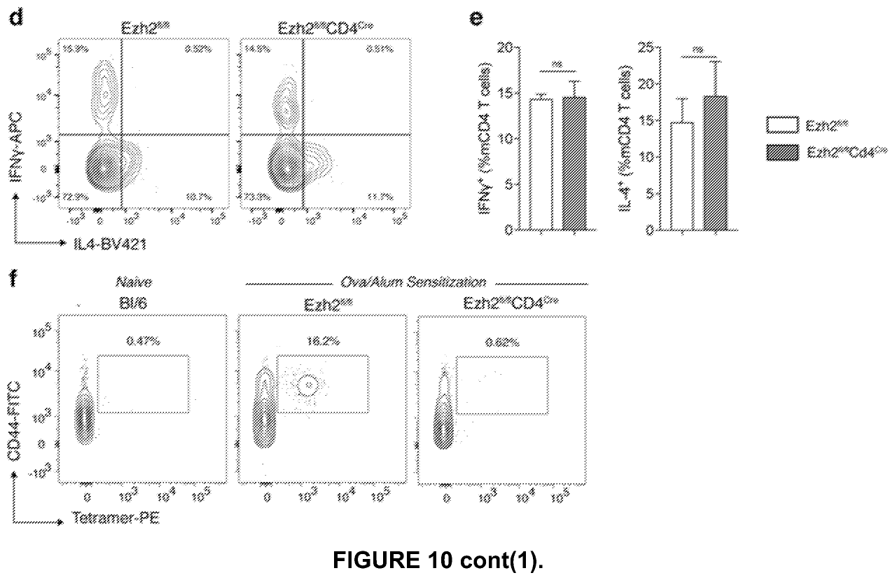

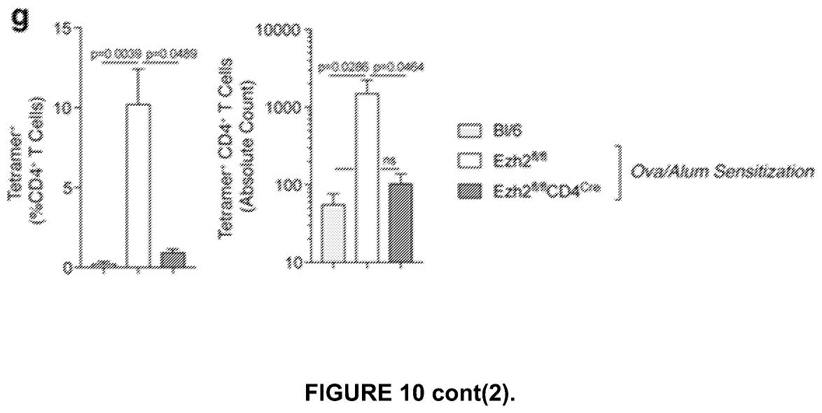

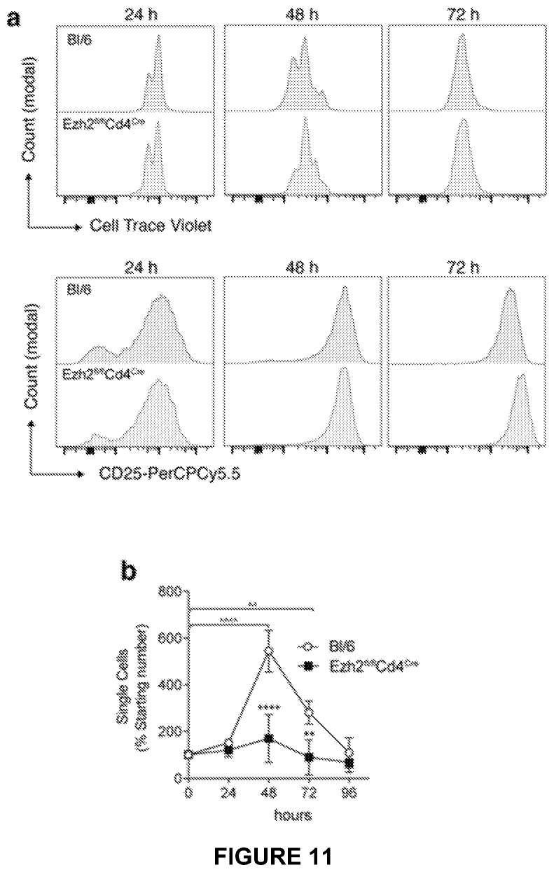

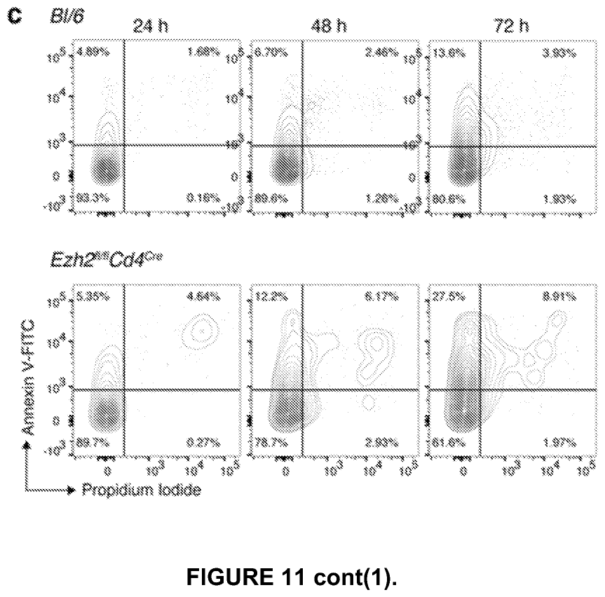

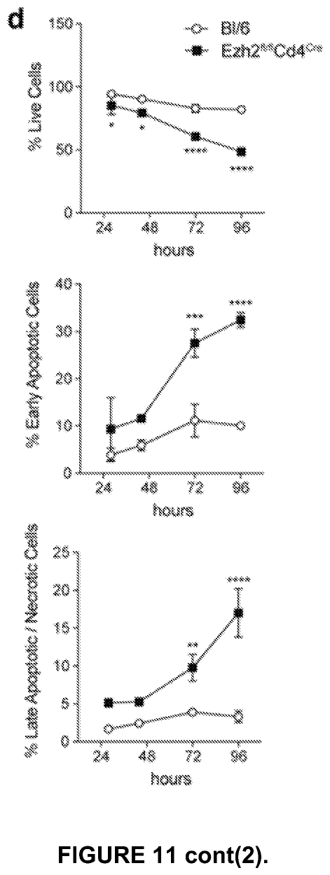

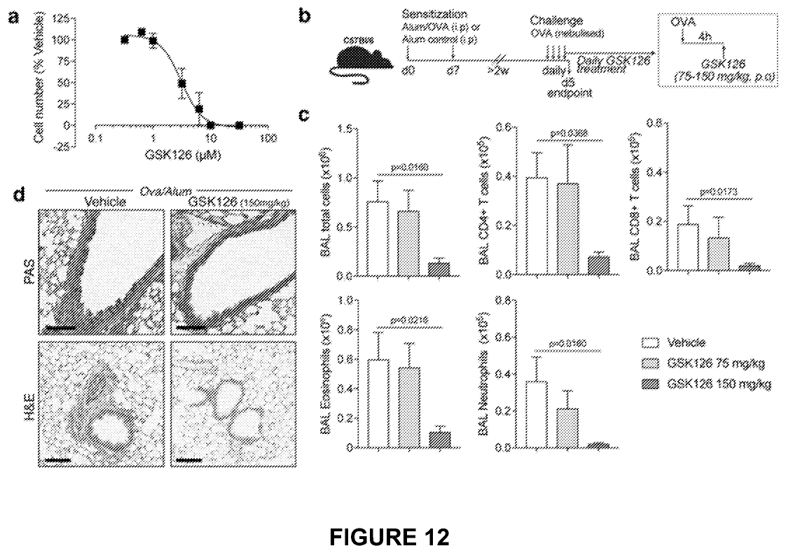

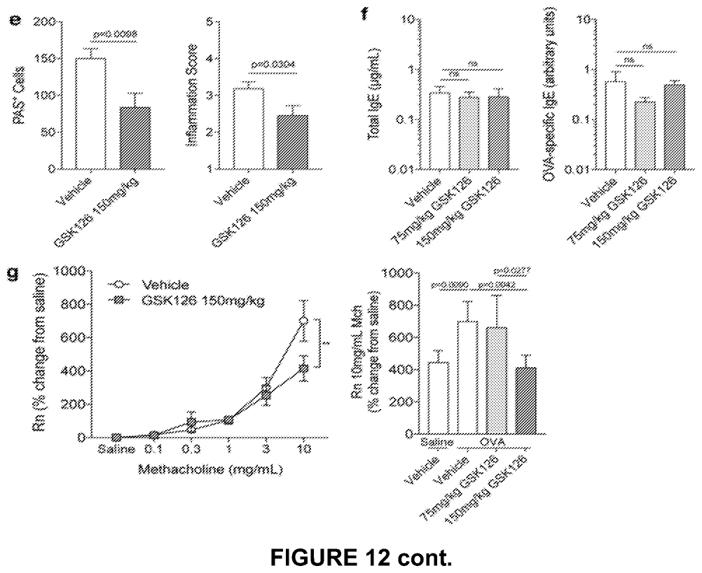

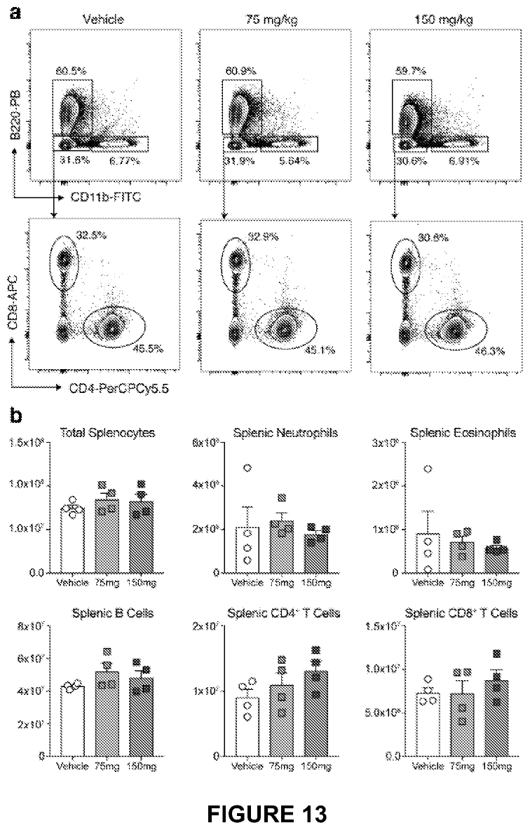

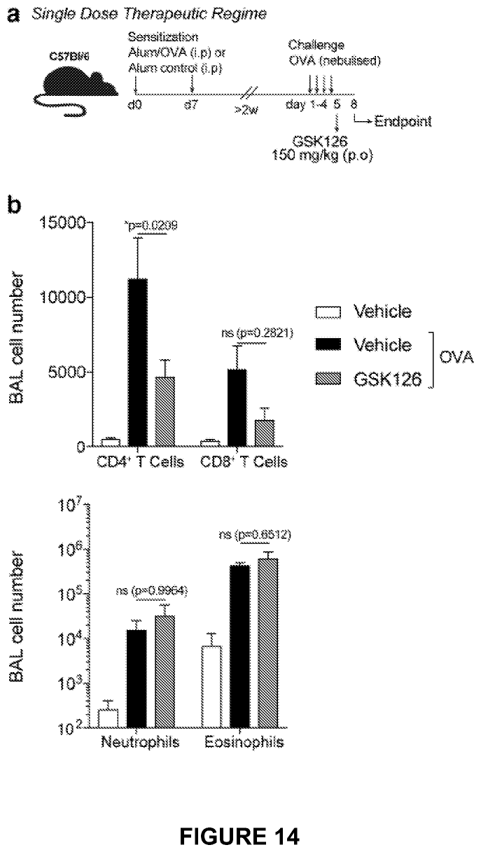

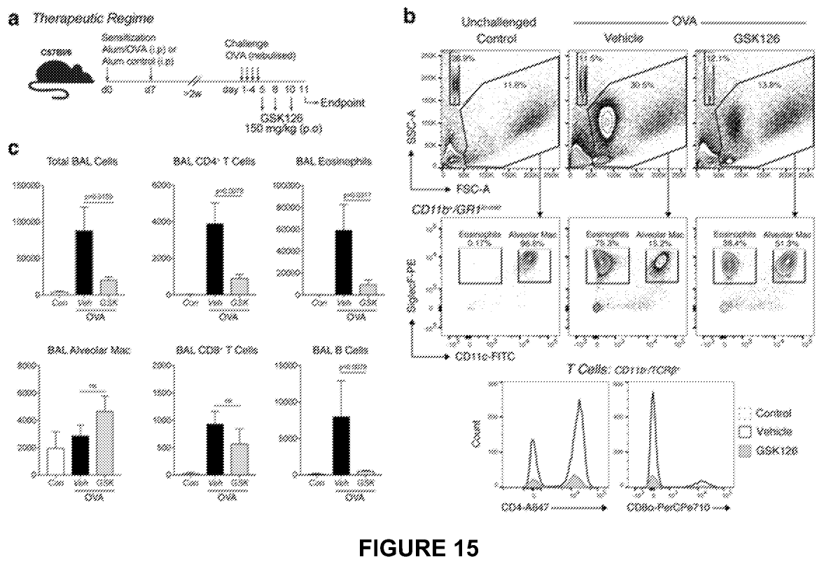

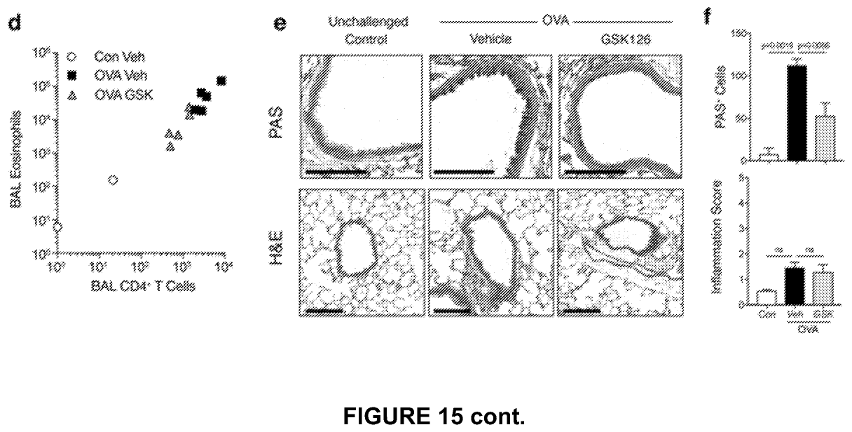

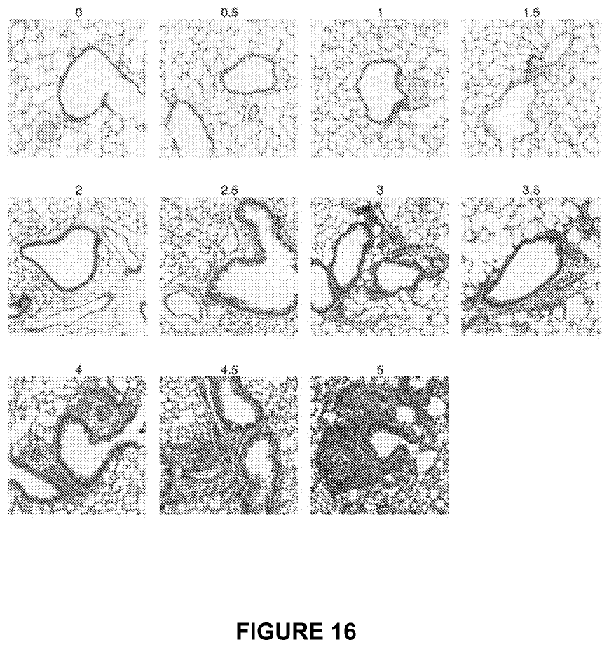

Activation-induced regulation of these chromatin-modifying genes at the protein level was then investigated. Wild-type mouse CD4⁺ T cells were activated and immunoblotting performed to assess the levels of each protein for which reliable antibodies were available. In line with the microarray analysis substantially increased protein levels of the PRC2 components Ezh2 and Suz12 in cells activated for 24 hours (FIG. 2b & c) were observed. The inventors also observed increased levels of HP1a (Cbx5) and HP1β (Cbx1) protein levels, whereas the expression of the HP1 binding factor, transcription intermediary factor 1-beta (TIF1β encoded by Trim28) was unaltered at this time-point (FIG. 2b & c).

LaminB1 was used as a nuclear loading control and was found to be lower in activated samples (FIG. 2b), suggesting the nuclear up-regulation of the protein components of these epigenetic silencing pathways was underestimated. Based on this data, the inventors next chose to explore the importance of the PRC2 and co-repressors of the Suv39h-H3K9me3 pathway in allergic asthma by specifically inactivating them in T cells.

### Example 2 Auxiliary Components of the Suv39h-H3K9Me3-HP1 Pathway are not Required for the Development of Allergic Inflammation

Given a number of molecules in the Suv39h1-H3K9me3-HP1 pathway were up regulated after T cell activation (FIG. 2), the roles of HP1α, HP1β and TIF1β in the allergic response was examined. Previously, the inventors had implicated HP1α (Cbx5) but not HP1γ (Cbx3) in Th2 cell stability (Allan et al. (2012) Nature 487, 249-253), making HP1γ an unlikely candidate. Therefore, mice that had exons of Cbx5 (HP1α), Cbx1 (HP1β) or Trim28 (TIF1β) flanked by loxP sequences were crossed to transgenic mice expressing cre recombinase under the control of the Cd4 promoter. In order to confirm the loss of the gene product specifically in T cells in each of these strains, splenic B cells (B220⁺) and CD4⁺ T cells (TCRβ⁺, CD4⁺) (FIG. 3a), were sorted and examined HP1α, HP1β and TIF1β protein expression by western blotting (FIG. 3b). Further analysis revealed that all these mice displayed normal T cell development (data not shown). To test the ability of these mice to mount an allergic response they were subjected to the classical ovalbumin (OVA) challenge model prior to comprehensive analysis of the cellular and cytokine composition of the lung environment. In this model mice are first sensitized to OVA in the presence of the adjuvant aluminium hydroxide (Alum) prior to challenge with aerosolized OVA. Despite lacking these molecules, surprisingly, these mice had normal cellular infiltrate and developed allergic pathology equivalent to their littermate counterparts (FIG. 4a-d). In order to exclude confounding influences from background effects, bronchoalveolar lavage (BAL) on naïve Cbx5^{fl/fl} Cd4^{Cre}, Cbx1^{fl/fl} Cd4^{Cre} and Trim28^{fl/fl} Cd4^{Cre} mice and their floxed counterparts was performed no difference was found in individual leukocyte populations within the lung of deleted or floxed mice, with alveolar macrophages forming the largest population of cells, as expected (FIG. 5). Bioplex cytokine analysis of the acellular lavage fluid from OVA-exposed mice showed some alterations in cytokine levels (FIG. 4e), particularly IL-4 and IL-5 levels in Cbx5^{fl/fl}Cd4^{Cre} mice, however this did not result in decreased inflammatory infiltration or decreased lung pathology (FIG. 4a-d). Thus, HP1α, HP1β and TIF1β molecules are not critical for T cells to drive allergic inflammation.

### Example 3 T Cells Rely on PRC2 Components to Drive Allergic Inflammation

The inventors then examined the role of Ezh2 in the allergic response as it was the most up regulated gene and protein following T cell activation. They bred mice in which the exons of Ezh2 (Su et al. (2003) Nat Immunol 4:124-131), were flanked by loxP sequences to transgenic mice expressing cre recombinase under the control of the Cd4 promoter (Lee et al. (2001) Immunity 15:763-774). This results in efficient deletion of Ezh2 in the T cell lineage and the mice display normal T cell development, but have alterations in CD8 memory phenotypes and NKT cell expansion (Vasanthakumar et al. (2017) EMBP Rep 18:619-631). As a control, some mice were exposed to alum alone prior to OVA challenge to test for any pre-existing or spontaneous immune reaction driven by the gene deficiency (FIG. 6a). First, the BAL infiltrate was examined by flow cytometry. Ezh2^{fl/fl} Cd4^{Cre} mice were completely protected from eosinophil, neutrophil and T cell infiltration into the airways following OVA challenge (FIG. 6b-d). The FSC vs SSC profiles of the neutrophil, eosinophil, alveolar macrophage and T cell populations gated in (FIG. 6c) are seen in (FIG. 7). Each of these populations correspond to the major cellular populations visible on FSC vs SSC plots shown in (FIG. 6b). Cells recovered from these Ezh2^{fl/fl} Cd4^{Cre} mice were predominantly alveolar macrophages (FSC^{hi}, SSC^{hi}, CD11b⁺, CD11c⁺, SiglecF⁺), a resident, non-inflammatory cell population whose numbers were consistent across groups.

Next, histopathology following OVA challenge was examined in Ezh2^{fl/fl} Cd4^{Cre} and control mice. OVA-sensitized Ezh2^{fl/fl} Cd4^{Cre} mice were completely protected from OVA-induced increases in PAS⁺ mucous producing cells and lung tissue inflammation levels (FIGS. 6e and f), statistically indistinguishable from alum only sensitized control mice (FIGS. 6e and f), demonstrating that Ezh2 deletion in T cells can completely prevent lung inflammation and mucous hypersecretion, characteristic of pulmonary allergic disease. It was then investigated whether this protection from pulmonary inflammation translated to improvements in lung function. Using the forced-oscillation technique coupled with inhaled bronchoconstrictor agent (methacholine 0.1-30 mg/mL), it was found that OVA-induced hyper-responsiveness seen in Ezh2^{fl/fl} control mice was absent in Ezh2^{fl/fl} Cd4^{Cre} mice whose airways resistance (Rn) was indistinguishable from unchallenged Bl/6 wildtype mice (FIG. 6g).

The inventors also used an alternative model of allergic inflammation representing a more physiologically relevant allergen, house dust mite (HDM) extract (FIG. 8a). In line with the previous findings using the OVA model, Ezh2^{fl/fl} Cd4^{Cre} mice were completely protected from the development of HDM-induced allergic inflammation (FIG. 8b-3-e). HDM-induced increases in BAL leukocytes in Ezh2^{fl/fl} control mice was significantly reduced in Ezh2^{fl/fl} Cd4^{Cre} mice, with the cell numbers recovered from Ezh2^{fl/fl} Cd4^{Cre} lavage indistinguishable from PBS exposed controls (FIG. 8c). The cellular infiltrate was predominantly eosinophils and T cells, with a lack of robust neutrophil accumulation in this model (FIG. 8c). Lung histopathology showed reduced mucous producing cells and decreased lung inflammation levels in HDM challenged Ezh2^{fl/fl} Cd4^{Cre} mice compared with Ezh2^{fl/fl} controls (FIGS. 8d and e), consistent with previous results using the OVA model. Overall these results suggest that the presence of Ezh2 in T cells is absolutely required for allergic responses in the airways.

In order to confirm that Ezh2 was mediating allergic inflammation through its canonical role as the enzymatic component of the PRC2 complex, the inventors separately inactivated the non-redundant core components of PRC2, Suz12 and Eed, specifically in T cells. Importantly, EED was the second most highly up-regulated repressive component after EZH2 in the T cell activation human microarray dataset (FIG. 2a), and Suz12 was highly up-regulated in mouse T cells at a protein level (FIG. 2c). Using mice deficient in either Suz12 (Lee et al. (2015) Blood 126:167-175) or Eed (Xie et al. (2014) 14:68-80) in the T cell compartment (Suz12^{fl/fl} Cd4^{Cre}, Eed^{fl/fl} Cd4^{Cre}), the inventors confirmed the absolute requirement for these molecules in the generation of allergic histopathology (FIG. 9a), eosinophilia (FIG. 9b, c) and cytokine production (FIG. 9d) in the lungs of OVA challenged mice. Thus, the PRC2 pathway is critical to development of T cell allergy.

### Example 4 Ezh2 is Required to Generate Antigen-Specific Memory

The mechanism by which deletion of PRC2 components in T cells prevents allergic inflammation was next investigated. First, cytokine levels in the acellular BAL fluid following OVA-challenge was examined and it was found almost all OVA-induced cytokines including the Th2 cytokines IL-4, IL-5 as well as pro-inflammatory cytokines IL-6 and KC, the murine CXCL8 homologue, were reduced to baseline levels in the lavage fluid from Ezh2^{fl/fl} Cd4^{Cre} mice (FIG. 10a). Importantly, loss of Ezh2 did not result in a skewing towards Th1 cytokines in the BAL fluid with IFNγ levels consistent across all groups (FIG. 10b).

Next, the titres of OVA-specific and total IgE in serum from these mice was quantified following OVA challenge. It was found that Ezh2^{fl/fl} Cd4^{Cre} mice lacked any detectable OVA-specific IgE above the background of the assay (FIG. 10c), despite normal levels of serum total IgE (FIG. 10c). Detectable levels of OVA-specific IgE were found in the serum from Bl/6 control mice following OVA challenge as expected (FIG. 10c).

To examine Th2 development in mice lacking Ezh2 in T cells, splenocytes from Ezh2^{fl/fl} Cd4^{Cre} mice and Ezh2^{fl/fl} control mice were isolated 10 days following initial sensitization to OVA. Cytokine production was stimulated by PMA/Ionomycin stimulation for 5 h in the presence of Golgistop protein transport inhibitor for the final 2 h prior to assessing intracellular IFNγ and IL-4 cytokine accumulation by flow cytometry. It was found that within the antigen-experienced CD4⁺ T cell compartment (CD44⁺), frequencies of IFNγ and IL-4 cytokine producing cells did not differ between Ezh2^{fl/fl} Cd4^{Cre} mice and Ezh2^{fl/fl} control mice (FIG. 10d, e), suggesting that Th2 cells can develop in the absence of Ezh2.

Examination of the frequency of OVA-specific CD4⁺ T cells in the spleen and peripheral lymph nodes of Ezh2^{fl/fl} Cd4^{Cre} and control floxed mice 10 days following initial OVA sensitization revealed a dramatic reduction in the number of OVA-specific CD4⁺ T cells in Ezh2^{fl/fl} Cd4^{Cre} mice compared with Ezh2^{fl/fl} mice (FIGS. 10f and g). Indeed, the frequency of tetramer⁺ CD4⁺ T cells from Ezh2^{fl/fl} Cd4^{Cre} mice was statistically indistinguishable from background levels of non-sensitized Bl/6 controls (FIGS. 10f and g). These data, together with the lack of OVA-specific IgE, suggested that Ezh2 is required to generate antigen-specific memory.

The inventors hypothesized that the failure to generate antigen-specific CD4⁺ T cells in the absence of Ezh2 was due to a defect in T cell proliferation. Surprisingly, when CD4⁺ T cells from Ezh2^{fl/fl} Cd4^{Cre} and Bl/6 control mice were labelled with Cell Trace Violet (CTV) dye and activated in vitro using anti-CD3 and anti-CD28 antibodies, similar degrees of progressive dye dilution in both Ezh2^{fl/fl} Cd4^{Cre} and Bl/6 control mice was seen, as well as similar up-regulation of the activation marker CD25 (FIG. 11a), suggesting intact activation responses and normal cell division. However, when cell numbers were measured over time, a profound defect was observed in clonal expansion of cells from Ezh2^{fl/fl} Cd4^{Cre} mice (FIG. 11b). Quantitation of cell death processes using Annexin-V and propidium iodide staining following cell activation showed a large increase in the proportion of Ezh2^{fl/fl} Cd4^{Cre} cells undergoing apoptosis compared to wildtype cells (FIGS. 11c and d). This premature cell death in cells lacking PRC2 function therefore prevents a robust T cell expansion, thereby curtailing inflammatory CD4⁺ T cell response.

### Example 5 Inhibition of Ezh2 Suppresses T Cell Expansion and Allergic Responses In Vivo

Having shown that mice deficient in PRC2 components do not develop allergic inflammation, the Ezh2 small molecule inhibitor GSK126 was used to test its therapeutic potential for allergic disease. GSK126 is a selective, S-adenosyl-L-methionine-competitive small molecule inhibitor of Ezh2 methyltransferase activity that has been shown to suppress the growth of tumours in mouse models (McCabe et al. (2012) Nature 492:108-122). Given that activation of T cells lacking Ezh2 function resulted in induction of apoptosis and a diminished immune response, it was hypothesized that small molecule inhibition of Ezh2 will similarly prevent T cell expansion. To test this, activated wild type CD4+ T cells were activated in vitro using anti-CD3 and anti-CD28 antibodies, and exposed to GSK126 for three days. A dose-dependent effect on the number of cells recovered in the culture compared to vehicle control was observed (FIG. 12a).

Ezh2 activity was the inhibited in vivo by administering GSK126 by oral gavage on each day of OVA aerosol challenge, 4 h after the OVA exposure (FIG. 12b). OVA challenge of vehicle treated control mice resulted in the accumulation of predominantly eosinophils in the airspace, along with smaller numbers of neutrophils and T cells (FIG. 12c). The 150 mg/kg dose of GSK126 resulted in a dramatic reduction in each of these individual populations as well as total BAL leukocytes (FIG. 12c). PAS⁺ mucous-producing epithelial cells and levels of inflammation in the lung tissue were similarly reduced in the airways of mice treated with 150 mg/kg GSK126 (FIG. 12d, e). The 75 mg/kg dose of GSK126 had no effect on BAL cells (FIG. 12c), PAS staining (data not shown), or lung tissue inflammation (data not shown) compared with vehicle treated mice. The administration of this drug did not lead to a global loss of immune cells as the numbers of lymphocytes and granulocytes in the spleen were unchanged (FIG. 13). The concentration of OVA-specific IgE and total IgE was also unchanged in the serum of mice treated with GSK126 (FIG. 12f), suggesting that inhibition of Ezh2 is a viable therapeutic strategy despite existing humoral and cellular memory in allergic individuals. Importantly, the 150 mg/kg dose of GSK126 also provided complete protection against OVA-induced airway hyperresponsiveness (FIG. 12g).

### Example 6 Inhibition of Ezh2 Suppresses Established Inflammation

In order to further test the therapeutic potential of Ezh2 inhibition in established inflammation, a single-hit experiment was performed whereby allergic inflammation was established in a standard OVA model of systemic sensitization followed by daily nebulized OVA exposure for 4 days. On the fifth day, rather than assessing allergic inflammation as had been done previously, a single dose of GSK126 (150 mg/kg) was administered by oral gavage and then assessed bronchoalveolar inflammation 3 days later (FIG. 14a). This single dose given following established inflammation resulted in reduced BAL CD4⁺ T cell numbers and did not alter granulocyte numbers, suggesting a selective effect on actively dividing, allergy-promoting T cells in the lung microenvironment (FIG. 14b).

The therapeutic regime was then extended to three doses (day 5, 8 and 10) after establishing inflammation by OVA challenge on days 1-4, and airway inflammation assessed on day 11 (FIG. 15a). Inhibition of Ezh2 in this therapeutic regime resulted in reduced airway inflammation (FIG. 15b), with total BAL cells, CD4⁺ T cells eosinophils and B cells all significantly reduced following GSK126 treatment (FIG. 15b, c). Interestingly, numbers of BAL eosinophils recovered was highly correlated with numbers of BAL CD4⁺ T cells (FIG. 15d); Pearson R²=0.9261, P<0.0001). Examination of lung histopathology revealed reduced PAS⁺ mucous-producing cells following therapeutic GSK126 treatment (FIG. 15e, f). However, lung inflammation levels had ablated by this point (7 days after final OVA challenge) and all groups were statistically indistinguishable (FIG. 15e, f). Overall, this data validates the gene inactivation experiments and shows that inhibiting the enzymatic activity of Ezh2 represents a novel strategy for suppressing allergic responses, even in established inflammation.

### Remarks

Targeting the enzymes that are responsible for laying down epigenetic modifications to chromatin has recently become possible with the design of several inhibitory drugs (Kelly et al., (2010) Nat Biotechnol. 28:1069-1078; Tough et al., (2016) Nat Rev Drug Discv 15:835-853). Concerns that targeting part of the epigenetic machinery would have widespread deleterious effects have been alleviated by clinical studies demonstrating that selective small molecule inhibitors that target individual epigenetic enzymes are well tolerated in cancer patients including many that target EZH2 (Gulati et al., (2018) leuk Lymphoma 59:1574-1585; Tough et al., 2016 supra). Indeed, the inventors did not observe any systemic effects at the doses of GSK126 used. The present data supports a selective effect of Ezh2 inhibition on rapidly dividing cells, such as CD4⁺ T cells mounting an allergic response, rather than terminally differentiated and relatively transcriptionally silent granulocyte populations. This suggests that while adaptive responses may be somewhat blunted through Ezh2 inhibition, innate responses would remain intact. Furthermore, it is plausible that careful balancing of Ezh2 inhibition levels may be able to provide protection from unwanted allergic/autoimmune activation, whilst retaining sufficient T cell response when antigen-load is high. It is therefore considered that Ezh2 inhibition represents a viable therapeutic strategy to target the lymphocytes that drive lung inflammation.

The data using therapeutic regimes of GSK126 provides further evidence that Ezh2 inhibition is indeed a viable therapeutic strategy for diseases such as allergic asthma. Whilst genetic deletion of Ezh2 prevented the development of antigen-specific CD4⁺ T cells and antigen-specific IgE, thereby precluding a subsequent immune response to ovalbumin or house dust mite challenge, GSK126 treatment was able to reduce airway inflammation and hyperresponsiveness even when antigen-specific memory was well established. The therapeutic efficacy of Ezh2 inhibition could be seen when this drug was administered in the context of ongoing established inflammation. Interestingly, the observed correlation between airway eosinophilia and CD4⁺ T cell numbers reflects what is seen in human asthmatic disease (Walker et al., (1991b) J Immunol. 146:1829-1835) and importantly also correlates with asthma severity (Walker et al., (1991a) J Allergy Clin Immunol 88:935-942). These results provide a rationale to expect utility of an Ezh2 inhibitor as a therapeutic in uncontrolled disease, and not just as a prophylactic treatment.

Given the tolerability of Ezh2 inhibitors in human studies, even when dosed through systemic routes, the transition from oncology to treatments for inflammation would be expected to be smooth and rapid. Especially given the dosing regimen used for the treatment of allergic inflammation would be expected to be much more restricted, than that for cancer, and ideally organ-restricted (i.e. to the lungs for allergic asthma).

1. A method for preventing, reducing one or more symptoms of, or treating inflammation in a subject, comprising administering to the subject an inhibitor of the polycomb repressive complex 2 (PRC2) methyltransferase enhancer of zeste homolog 2 (Ezh2).

2. The method according to claim 1, wherein the inflammation is allergic inflammation.

3. The method according to claim 1, wherein the inflammation is lymphocyte-driven inflammation.

4. The method according to claim 3, wherein the inflammation is Th cell driven.

5. The method according to claim 3, wherein the lymphocyte-driven inflammation is chronic obstructive pulmonary disease (COPD), autoimmune disease or diabetes.

6. The method according to claim 1, wherein the inflammation is non-allergic asthma.

7. The method according to claim 2, wherein the allergic inflammation is any disorder which is initiated by an allergen.

8. The method according to claim 2, wherein the allergic inflammation is selected from the group consisting of allergic asthma, atopic dermatitis, allergic rhinitis (hay fever), urticaria (hives), food allergies, drug allergies, anaphylaxis and ocular allergic disorders.

9. The method according to claim 1, wherein the method reduces the effect of one or more symptoms of allergic inflammation selected from the group consisting of itchiness, running nose, sneezing, watery eyes, bronchoconstriction, hives, airway inflammation, anaphylaxis, and dermatitis.

10. The method according to claim 1, wherein the Ezh2 inhibitor is selected from the group consisting of immunoglobulins (e.g. antibodies or antigen-binding fragments thereof), oligonucleotides, ribozymes, aptamers, siRNAs, anti-sense molecules, peptides and drugs (e.g. small molecule inhibitors).

11. The method according to claim 1, wherein the Ezh2 inhibitor is administered in combination with an asthma and/or allergy medicament.

12. The method according to claim 1, wherein the subject is one at risk of developing an inflammation disorder.

13. The method according to claim 1, wherein the subject has an allergic inflammation disorder.

14. The method according to claim 1, wherein the subject has asthma.

15. The method according to claim 1, wherein the subject is wild-type for the ezh2 gene.

16. The method according to claim 1, wherein the subject is mutant for the ezh2 gene.

17. (canceled)

18. (canceled)
